(19) United States
(12) Patent Application Publication
(10) Pub. No.: US 2004/0175423 A1
(43) Pub. Date: Sep. 9, 2004

(54) Sustained release formulation of N- (2-propylpentanoyl) glycinamide and related compounds

(76) Inventors: Daniella Licht, Ramat Ilan Givat Shmuel (IL); Ioana Lovinger, Lod (IL); Suher Abd-Elhai, Tira (IL); Mazzi Dagan-Lion, Modiin (IL); Adrian Gilbert, Rananna (IL); Noa Leibovitch, Ramat-Ha Sharon (IL); Sasson Cohen, Tel Aviv (IL); Ruth Levy, Tel-Aviv (IL)

Correspondence Address: John P. White, Cooper & Dunham LLP, 1185 Avenue of the Americas, New York, NY, 10036, US

(21) Appl. No.: 10/773,472

(22) Filed: Feb. 5, 2004

Related U.S. Application Data
(60) Provisional application No. 60/445,328, filed on Feb. 5, 2003.

(51) Int. Cl. A61K 31/16; A61K 31/19; A61K 9/22

(52) U.S. Cl. 424/468; 514/557; 514/616

(57) ABSTRACT

The subject provides a sustained release tablet comprising the following components: a) a uniform admixture of an active ingredient selected from the group consisting of valproic sodium acid, a pharmaceutically acceptable salt or ester of valproic acid, divalproex sodium, valpromide and a compound having the structure:

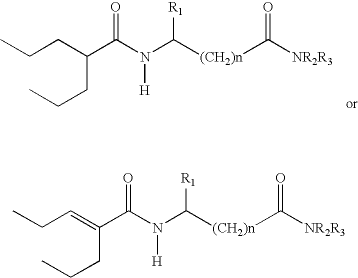

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and a binder, and b) a hydroxypropylmethyl cellulose, a process for manufacturing the tablet and a method of treating neuropathic pain, epilepsy, mania in bipolar disorder, a headache disorder, pain or of effecting pain prophylaxis in a subject.

This application claims the benefit of U.S. Provisional Application No. 60/445,328, the entire contents of which are hereby incorporated by reference.

Throughout this application, various publications are referenced by full citations. The disclosures of these publications in their entireties are hereby incorporated by reference into this application in order to more fully describe the state of the art as known to those skilled therein as of the date of the invention described and claimed herein.

### BACKGROUND OF THE INVENTION

Pain is considered to play a basic physiological role in the detection and localization of tissue damage or potentially damaging physiological processes. Pain has been broadly classified as somatogenic, where a physiological explanation can be found, or psychogenic, where the physiological explanation is not known (The Merck Manual of Diagnosis and Therapy, 16^{th} Ed., pp. 1407-1426; PCT International Publication No. WO 02/13766 A2). An example of somatogenic pain is neuropathic pain.

Neuropathic pain is a category of pain which includes several forms of non-nociceptive chronic pain, which result from dysfunction of nervous rather than somatic tissue. The majority of non-nociceptive chronic pains, in terms of either syndromes or cases, follow at various times after damage to either central or peripheral nervous tissue. Diagnosis of most of these syndromes and cases reveals a dependence on abnormal spatial and temporal summation of natural somatic stimulation in the spinal cord and independence from somatic disease and peripheral sympathetic nervous system activity. The scientific pain research community defines this kind of pain as centrally mediated neuropathic pain and recognizes mechanistic, diagnostic, and therapeutic commonalities among pains of this class and differences between these and other syndromes.

Neuropathic pain can be defined as pain deriving from damage to or inflammation of central or peripheral nervous system tissue. Examples of pain syndromes of this class include post herpetic neuralgia, neuritis, temporomandibular disorder, myofascial pain, back pain, pain induced by inflammatory conditions. Neuropathic pain may occur in all body regions. For example, neuropathic pain may originate from the dental region.

Burn injury also often leads to neuropathic hyperalgesia in the affected body area. Neuralgia is characterized, in its acute phase, by intraneural inflammation, which can cause damage to primary afferent axons, thus inducing neuropathic pain. Neuropathic pain may also be induced by diabetic conditions (diabetic neuropathy). Neuropathy of primary afferent axons in long nerves is found in diabetic patients. Nociceptor sensitization may ensue (U.S. Pat. No. 6,054,461).

Pain can be both chronic and acute, and can also be evoked by noxious stimuli, referred to as hyperalgesia, or by non-noxious stimuli, referred to as allodynia (Attal, N. “Mechanism of action and rationale for use of antiepileptic drugs” (1999) in International Congress and Symposium Series 241 The Royal Society of Medicine Press, Limited Ed. J M Pellock). Allodynia and hyperalgesia can have mechanical causes (dynamic or static), or a thermal cause. Examples of neuropathic pain include all the painful peripheral neuropathies and specifically diabetic peripheral neuropathy, postherpetic neuralgia, and trigeminal neuralgia. Trigeminal neuralgia, for example, is the most common neuralgic syndrome in the elderly.

Other types of somatogenic pain that may have neuropathic components include cancer pain, postoperative pain, lower back pain, complex regional pain syndrome, phantom pain, HIV pain, arthritis (osteo-arthritis and rheumatoid arthritis) pain and migraines.

Pain may also be a symptom of headache disorders. Migraines constitute one of the four major categories of primary headaches (International Headache Society, 1988; Silberstein, S. D. et al. Headache in Clinical Practice, (1998) Pub. Isis Medical Media, Oxford). The other three types of primary headaches are tension-type, cluster and a miscellaneous-type (Id.). One current view is that there is a continuous spectrum of headache severity ranging from mild tension headaches to severe migraines. Others consider tension headaches and migraines to be distinct entities.

Neuropathic pain conditions are characterized by hyperesthesia (enhanced sensitivity to a natural stimulus), hyperalgesia (abnormal sensitivity to pain), allodynia (widespread tenderness, characterized by hypersensitivity to tactile stimuli), and/or spontaneous burning pain.

The initial drug of choice for treating trigeminal neuralgia is carbamazepine. For other types of pain, such as postherpetic neuralgia and painful diabetic neuropathy, amitriptyline is most commonly used.

Drugs used in the treatment of headache disorders such as migraines originate from a broad range of different drug categories. These include: 5-hydroxytryptamine agonists (5-HT₁ agonists), dihydroergotamine, ergotamine, anti-emetics, anxiolytics, non-steroidal anti-inflammatory drugs, steroids, major tranquilizers, narcotics, beta-blockers, calcium channel blockers, anti-depressants, and anti-epileptic drugs. However, not all of the drugs in these categories are truly effective. There is still a need for more efficacious drugs, as well as a need for antimigraine treatments with fewer side effects.

Epilepsy is an ancient disease, which affects about 1% of the global population. Despite the progress made in antiepileptic drug therapy, there are still many patients who continue to suffer from uncontrolled seizures and medication toxicity. At present, only the following 4 major antiepileptic drugs are in use: phenobarbital, phenyloin, carbamazepine and valproic acid. About 25% of the patient population is not seizure-free while treated with these medications (both mono and polytherapy) even when diagnosis and therapy is optimal (“Sustained Release Formulations of Antiepileptics” Clin. Pharmacokinet. (1992) 22(1): 11-24).

Pharmacological activity in general and antiepileptic activity in particular, correlate better with the concentration of a drug in the blood (or in some other biophase) than with the administered dose. This phenomenon is due, in part, to variability in drug absorption and disposition between and within individuals, particularly when the drug is given orally.

Optimizing drug therapy aims at achieving and maintaining therapeutic and safe drug concentration in the blood. In order to achieve this goal, it would be advantageous, and probably more convenient, that the patient receive a once- or twice-daily dosage regimen (Ballard 1978; Silber et al. 1987, Welling 1983).

N-(2-Propylpentanoyl)glycinamide is an anti-epilepsy and anti-pain drug which has the structure:

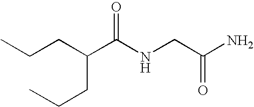

and can be prepared as disclosed by Bialer et al. in U.S. Pat. No. 5,585,358. U.S. Pat. No. 5,585,358 also describes a series of derivatives of valproic acid amides and 2-valproenic acid amides for the treatment of epilepsy and other neurological disorders.

Bialer et al. refer to the above compound as N-(2-n-Propylpentanoyl)glycinamide. However, in the present application, the compound is referred to as N-(2-Propylpentanoyl)glycinamide.

Published U.S. Patent Application No. US-2002-0052418-A1 discloses the use of N-(2-Propylpentanoyl)glycinamide and other derivatives of valproic acid amides and 2-valproenic acid amides for the treatment or prevention of pain and/or headache disorders.

U.S. Pat. No. 5,009,897, issued Apr. 23, 1991 discloses granules, suitable for pressing into tablets, the granules comprising a core of divalproex sodium and a coating of a mixture of a polymer and microcrystalline cellulose.

U.S. Pat. No. 4,913,906, issued Apr. 3, 1990, discloses controlled release dosage forms of valproic acid, its amide, or one of its salts or esters in combination with a natural or synthetic polymer, pressed into a tablet under high pressure.

U.S. Pat. No. 4,913,906 does not, however, disclose the use of hydroxypropylmethyl cellulose, or the use of two or more materials to achieve controlled release.

U.S. Pat. No. 6,419,953, issued Jul. 16, 2002, discloses controlled release formulations of valproic acid, its salt, divalproex sodium, or valpromide, comprising granules of the active ingredient, each granule containing the active compound, hydroxypropylmethyl cellulose and lactose, mixed with additional excipients. In U.S. Pat. No. 6,419,953, the hydroxypropylmethyl cellulose, if used, is part of each granule. U.S. Pat. No. 6,419,953 does not disclose compressing granules of active ingredient with hydroxypropylmethyl cellulose.

The subject invention provides a sustained release formulation of N-(2-Propylpentanoyl)glycinamide.

### SUMMARY OF INVENTION

The subject provides a sustained release solid dosage form comprising the following components:

a) a uniform admixture of:

(i) an active ingredient selected from the group consisting of valproic sodium acid, a pharmaceutically acceptable salt or ester of valproic acid, divalproex sodium, valpromide and a compound having the structure:

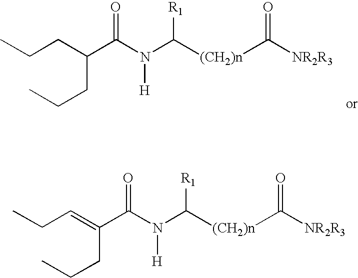

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a binder, and

b) a hydroxypropylmethyl cellulose.

The subject invention also provides a sustained release tablet comprising the following components:

a) a uniform admixture of:

(i) N-(2-Propylpentanoyl)glycinamide; and

(ii) a binder;

b) a hydroxypropylmethyl cellulose; and

c) a different hydroxypropylmethyl cellulose.

The subject invention also provides a hard compressed tablet comprising a uniform admixture of the following components:

a) N-(2-Propylpentanoyl)glycinamide;

b) a hydroxypropylmethyl cellulose; and

c) a different hydroxypropylmethyl cellulose.

The subject invention also provides a composition in granulate form comprising a uniform admixture of:

(i) an active ingredient selected from the group consisting of valproic sodium acid, a pharmaceutically acceptable salt or ester of valproic acid, divalproex sodium, valpromide and a compound having the structure:

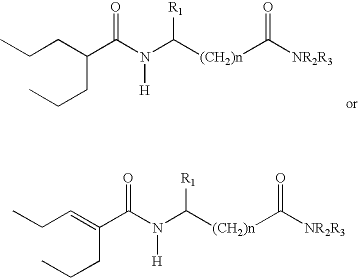

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a hydroxypropyl cellulose.

### DETAILED DESCRIPTION OF THE FIGURES

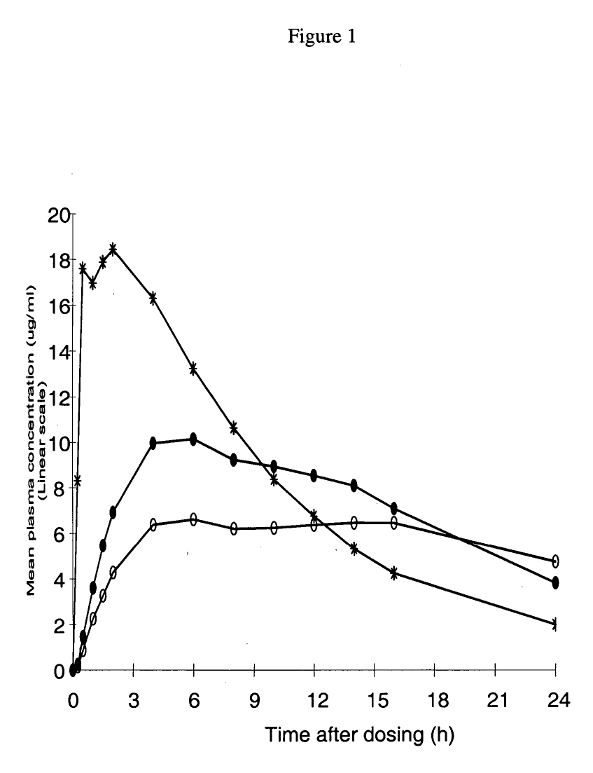

FIG. 1 shows mean plasma N-(2-propylpentanoyl)glycinamide concentrations following the administration of 2×500 mg N-(2-propylpentanoyl)glycinamide tablets (Formulation A), N-(2-propylpentanoyl)glycinamide tablets (Formulation B) and 2×500 mg N-(2-propylpentanoyl)glycinamide tablets (Formulation C) to eighteen healthy male Caucasian volunteers.

-●- Formulation A

-∘- Formulation B

-*- Formulation C

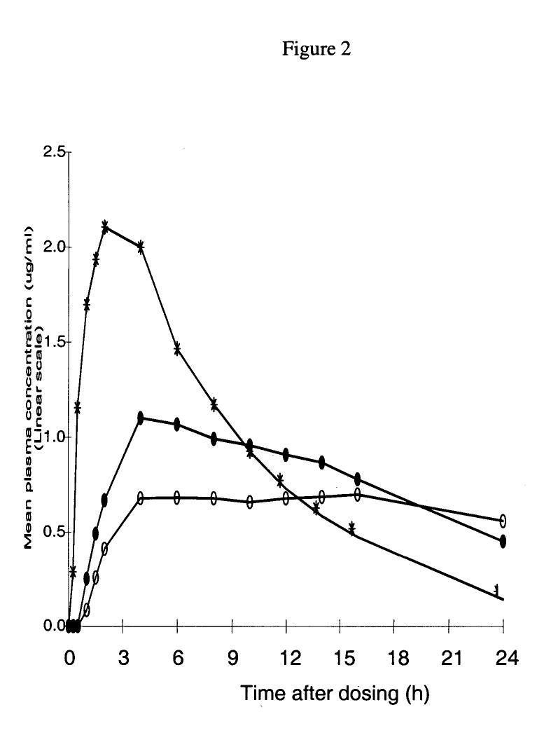

FIG. 2 shows mean plasma concentrations of N-(2-propylpentanoyl)glycine following the administration of 2×500 mg N-(2-propylpentanoyl)glycinamide tablets (Formulation A), 2×500 N-(2-propylpentanoyl)glycinamide tablets (Formulation B) and 2×500 mg N-(2-propylpentanoyl)glycinamide tablets (Formulation C) to eighteen healthy male Caucasian volunteers.

-●- Formulation A

-∘- Formulation B

-*- Formulation C

### DETAILED DESCRIPTION

The subject invention provides a sustained release solid dosage form comprising the following components:

a) a uniform admixture of:

(i) an active ingredient selected from the group consisting of valproic sodium acid, a pharmaceutically acceptable salt or ester of valproic acid, divalproex sodium, valpromide and a compound having the structure:

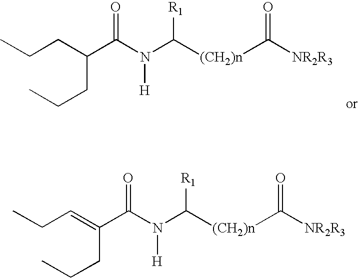

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a binder; and

b) a hydroxypropylmethyl cellulose.

In one embodiment, the solid dosage form is a tablet.

In another embodiment, the uniform admixture of component a) further comprises a filler.

In one embodiment, the filler comprises a microcrystalline cellulose.

In another embodiment, the hydroxypropylmethyl cellulose comprises 19%-24% by weight methoxyl substituent, 7%-12% by weight hydroxyproproxyl substituent and has a particle size distribution such that at least 99% of the hydroxypropylmethyl cellulose passes through a No. 40 US standard sieve, has an apparent viscosity of 78-117 millipascal-seconds (nominal value 98 mpa.s) by rotation and 80-120 cP (nominal value 100 cP) by Ubbelhode, at a concentration of 1% by weight in water at 20° C., and has a pH in the range 5.5-8.0.

In another embodiment, at least 90% of the hydroxypropylmethyl cellulose passes through a No. 100 US standard sieve.

In another embodiment, the solid dosage form further comprises as additional components a filler, a lubricant and a flow agent.

In another embodiment, the binder of component a)(ii) comprises hydroxypropyl cellulose.

In another embodiment, the solid dosage form further comprises a different hydroxypropylmethyl cellulose as a component.

In another embodiment, the solid dosage form further comprises as additional components a filler, a lubricant and a flow agent.

In another embodiment, the solid dosage form further comprises a different hydroxypropylmethyl cellulose.

In another embodiment, the different hydroxypropylmethyl cellulose comprises 19-24% by weight methoxyl substituent, 7-9% by weight hydroxypropoxyl substituent, has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C., has a pH in the range 5.5-8.0 and has a particle size distribution such that at least 99% of the hydroxypropylmethyl cellulose passes through a No. 40 US standard sieve.

In another embodiment, at least 90% of the hydroxypropylmethyl cellulose passes through a No. 100 US standard sieve.

In another embodiment,

the filler comprises a microcrystalline cellulose, anhydrous dicalcium phosphate, lactose, methylcellulose, carboxymethylcellulose, calcium carbonate, calcium sulfate kaolin, sodium chloride, powdered cellulose, sucrose, mannitol, starch, corn starch, various natural gums or a combination of two or more of the foregoing;

the lubricant comprises magnesium stearate! sodium stearyl fumarate, hydrogenated castor oil, hydrogenated soybean oil, polyethylene glycol or a combination of two or more of the foregoing; and

the flow agent comprises a colloidal fumed silica, or colloidal silicon dioxide.

In another embodiment,

the filler comprises a microcrystalline cellulose, anhydrous dicalcium phosphate, lactose or a combination of two or more of the foregoing;

the lubricant comprises magnesium stearate, sodium stearyl fumarate or a combination thereof; and

the flow agent comprises a colloidal fumed silica.

In another embodiment, the active ingredient is a compound having the structure:

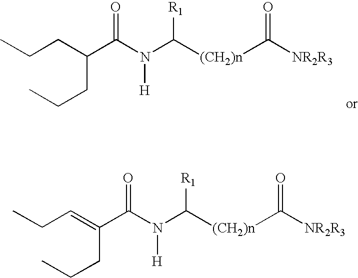

wherein R₁, R₂, and R₃ are independently the same or different and are hydrogen, a C₁-C₆ alkyl group, an aralkyl group, or an aryl group, and n is an integer which is greater than or equal to 0 and less than or equal to 3.

In a further embodiment, the active ingredient is N-(2-Propylpentanoyl)glycinamide.

In another embodiment, the above solid dosage form also comprises the following components:

a) a uniform admixture of:

(i) an active ingredient selected from the group consisting of valproic sodium acid, a pharmaceutically acceptable salt or ester of valproic acid, divalproex sodium, valpromide,

N-(2-Propylpentanoyl)glycinamide,

N-(2-propylpentanoyl)glycine-N′-methylamide,

N-(2-propylpentanoyl)glycine-N′-butylamide,

N-(2-propylpentanoyl)leucinamide,

N-(2-propylpentanoyl)alanine-N′-benzylamide,

N-(2-propylpentanoyl)alapinamide,

N-(2-propylpentanoyl)-2-phenylglycinamide,

N-(2-propylpentanoyl)threoninamide,

N-(2-propylpentanoyl)glycine-N′,N′-dimethylamide,

N-(2-propylpent-2-enoyl)glycinamide,

N-(2-propylpent-2-enoyl)alaninamide, and

N-(2-propylpent-2-enoyl)glycine-N′-methylamide; and

(ii) a binder, and

b) a hydroxypropylmethyl cellulose.

The subject invention also provides a sustained release solid dosage form comprising the following components:

a) a uniform admixture of:

(i) N-(2-Propylpentanoyl)glycinamide; and

(ii) a binder;

b) a hydroxypropylmethyl cellulose; and

c) a different hydroxypropylmethyl cellulose.

In one embodiment, the solid dosage form is a tablet.

In one embodiment, the solid dosage form comprises a filler, a lubricant and a flow agent as additional components and wherein the uniform admixture of component a) further comprises a filler.

In another embodiment,

the binder of component a) (ii) comprises hydroxypropyl cellulose;

the filler of component a) comprises a microcrystalline cellulose;

the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.;

the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;

the filler component comprises a microcrystalline cellulose, anhydrous dicalcium phosphate, lactose or a combination of two or more of the foregoing;

the lubricant component comprises magnesium stearate, sodium stearyl fumarate or a combination thereof; and

the flow agent component comprises a colloidal fumed silica.

In another embodiment, the solid dosage form comprises the following components:

a) a uniform admixture of:

(i) from 50 mg/solid dosage form to 1000 mg/solid dosage form of N-(2-propylpentanoyl)glycinamide,

(ii) from 1 mg/solid dosage form to 100 mg/solid dosage form hydroxypropyl cellulose; and

(iii) from 1 mg/solid dosage form to 200 mg/solid dosage form microcrystalline cellulose;

b) from 10 mg/solid dosage form to 300 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;

c) from 10 mg/solid dosage form to 300 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;

d) from 1 mg/solid dosage form to 300 mg/solid dosage form microcrystalline cellulose, anhydrous dicalcium phosphate, lactose or a combination of two or more of the foregoing;

e) from 0.1 mg/solid dosage form to 20 mg/solid dosage form of magnesium stearate, sodium stearyl fumarate or a combination thereof; and

f) from 0.1 mg/solid dosage form to 15 mg/solid dosage form a colloidal fumed silica.

In another embodiment, the solid dosage form comprises the following components:

a) a uniform admixture of:

(i) from 500 mg/solid dosage form to 850 mg/solid dosage form of N-(2-propylpentanoyl)glycinamide,

(ii) from 25 mg/solid dosage form to 75 mg/solid dosage form hydroxypropyl cellulose; and

(iii) from 50 mg/solid dosage form to 150 mg/solid dosage form microcrystalline cellulose;

b) from 100 mg/solid dosage form to 300 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;

c) from 20 mg/solid dosage form to 150 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;

d) from 20 mg/solid dosage form to 100 mg/solid dosage form microcrystalline cellulose, anhydrous dicalcium phosphate, lactose or a combination of two or more of the foregoing;

e) from 2 mg/solid dosage form to 20 mg/solid dosage form of magnesium stearate, sodium stearyl fumarate or a combination thereof; and

f) from 0.5 mg/solid dosage form to 5 mg/solid dosage form a colloidal fumed silica,

per 1 gram solid dosage form.

In one embodiment, at least 90% of the hydroxypropylmethyl cellulose of component b), of component c), or of both component b) and c) passes through a No. 100 US standard sieve.

In another embodiment,

the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

In another embodiment, the solid dosage form comprises the following components:

a) a uniform admixture of

(i) 500 mg/solid dosage form N-(2-Propylpentanoyl)glycinamide,

(ii) 50 mg/solid dosage form hydroxypropyl cellulose; and

(iii) 100 mg/solid dosage form microcrystalline cellulose;

b) 150 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;

c) 60 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;

d) 20 mg/solid dosage form lactose;

e) 4.5 mg/solid dosage form magnesium stearate; and

f) 1 mg/solid dosage form colloidal fumed silica.

In one embodiment, at least 90% of the hydroxypropylmethyl cellulose of component b), of component c), or of both component b) and c) passes through a No. 100 US standard sieve.

In another embodiment,

the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

The subject invention also provides a hard compressed tablet comprising a uniform admixture of the following components:

a) N-(2-Propylpentanoyl)glycinamide;

b) a hydroxypropylmethyl cellulose; and

c) a different hydroxypropylmethyl cellulose.

In one embodiment,

the hydroxypropylmethyl cellulose component b) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve; and

the hydroxypropylmethyl cellulose component c) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

In one embodiment, at least 90% of the hydroxypropylmethyl cellulose of component b), of component c), or of both component b) and c) passes through a No. 100 US standard sieve.

In another embodiment,

the hydroxypropylmethyl cellulose component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

the hydroxypropylmethyl cellulose component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

In another embodiment, the tablet further comprises a filler, lubricant and flow agent as additional components.

In another embodiment,

the filler comprises a microcrystalline cellulose, anhydrous dicalcium phosphate, lactose or a combination of two or more of the foregoing;

the lubricant comprises sodium stearyl fumarate; and

the flow agent comprises a colloidal fumed silica.

In another embodiment, the tablet comprises a uniform admixture of the following components:

a) from 100 mg/tablet to 1000 mg/tablet N-(2-Propylpentanoyl)glycinamide;

b) from 10 mg/tablet to 300 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;

c) from 10 mg/tablet to 300 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;

d) from 1 mg/tablet to 300 mg/tablet a microcrystalline cellulose, anhydrous dicalcium phosphate, lactose or a combination of two or more of the foregoing;

e) from 0.1 mg/tablet to 20 mg/tablet sodium stearyl fumarate; and

f) from 0.1 mg/tablet to 15 mg/tablet a colloidal fumed silica.

In another embodiment, the tablet comprises a uniform admixture of the following components:

a) from 400 mg/tablet to 1000 mg/tablet N-(2-Propylpentanoyl)glycinamide;

b) from 100 mg/tablet to 300 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;

c) from 20 mg/tablet to 150 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;

d) from 10 mg/tablet to 60 mg/tablet a microcrystalline cellulose, anhydrous dicalcium phosphate, lactose or a combination of two or more of the foregoing;

e) from 2 mg/tablet to 20 mg/tablet sodium stearyl fumarate; and

f) from 5 mg/tablet to 15 mg/tablet a colloidal fumed silica,

per 1 gram tablet.

In another embodiment, the tablet comprises a uniform admixture of the following components:

a) 500 mg/tablet N-(2-Propylpentanoyl)glycinamide;

b) 150 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;

c) 60 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;

d) 20 mg/tablet lactose;

e) 10 mg/tablet sodium stearyl fumarate; and

f) 10 mg/tablet colloidal fumed silica.

The subject invention also provides a composition in granulate form comprising a uniform admixture of:

(i) an active ingredient selected from the group consisting of valproic sodium acid, a pharmaceutically acceptable salt or ester of valproic acid, divalproex sodium, valpromide and a compound having the structure:

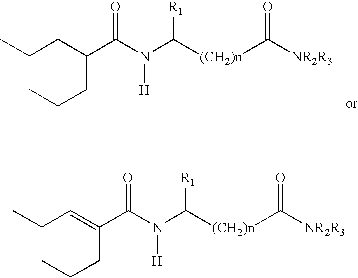

wherein R₁, R₂, and R₃ are independently the same or different and are hydrogen, a C₁-C₆ alkyl group, an aralkyl group, or an aryl group, and n is an integer

which is greater than or equal to 0 and less than or equal to 3; and

(ii) a hydroxypropyl cellulose.

In one embodiment of the composition, the active ingredient comprises a compound having the structure:

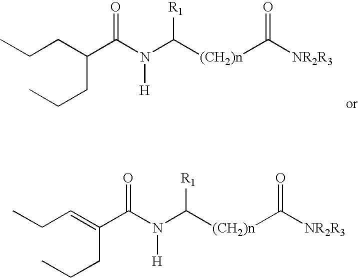

wherein R₁, R₂, and R₃ are independently the same or different and are hydrogen, a C₁-C₆ alkyl group, an aralkyl group, or an aryl group, and n is an integer which is greater than or equal to 0 and less than or equal to 3.

In another embodiment, the active ingredient comprises valproic sodium acid, a pharmaceutically acceptable salt or ester of valproic acid, divalproex sodium or valpromide.

The subject invention also provides a tablet comprising the above granulate as a component.

In one embodiment of the tablet, the granulate further comprises a filler.

In another embodiment, the tablet further comprises a hydroxypropylmethyl cellulose as a component.

In another embodiment, the tablet further comprises as additional components a filler, a lubricant and a flow agent.

In another embodiment, the tablet further comprises as additional components a filler, a lubricant and a flow agent.

In another embodiment, the tablet further comprises a different hydroxypropylmethyl cellulose as a component.

In another embodiment,

the hydroxypropylmethyl cellulose has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

In another embodiment, at least 90% of the hydroxypropylmethyl cellulose passes through a No. 100 US standard sieve.

In another embodiment,

the hydroxypropylmethyl cellulose has an apparent viscosity of 78-117 millipascal-seconds (nominal value 98 mPa.s) by rotation and 80-120 cP (nominal value 100 cP) by Ubbelhode, at a concentration of 1% by weight in water at 20° C.

In another embodiment,

the different hydroxypropylmethyl cellulose has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

In another embodiment, at least 90% of the hydroxypropylmethyl cellulose passes through a No. 100 US standard sieve.

In another embodiment,

the different hydroxypropylmethyl cellulose has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

In another embodiment, the filler in the granulate is a microcrystalline cellulose.

In another embodiment,

the filler comprises a microcrystalline cellulose, anhydrous dicalcium phosphate, lactose or a combination of two or more of the foregoing;

the lubricant comprises magnesium stearate, sodium stearyl fumarate or a combination thereof; and

the flow agent comprises a colloidal fumed silica.

The subject invention also provides a sustained release tablet comprising a compound having the structure:

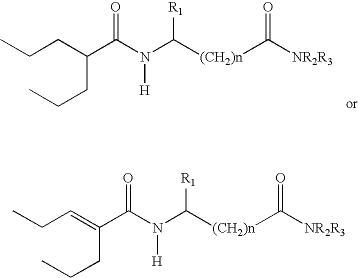

wherein R₁, R₂, and R₃ are independently the same or different and are hydrogen, a C₁-C₆ alkyl group, an aralkyl group, or an aryl group, and n is an integer which is greater than or equal to 0 and less than or equal to 3.

In one embodiment, the compound is N-(2-propylpentanoyl)glycinamide.

The subject invention also provides a method of treating neuropathic pain in a subject in need of such treatment comprising administering to the subject a therapeutically effective dose of any of the solid dosage forms or tablets of the invention in order to thereby treat the neuropathic pain in the subject.

The subject invention also provides a method of treating a headache disorder in a subject in need of such treatment comprising administering to the subject a therapeutically effective dose of any of the solid dosage forms or tablets of the invention in order to thereby treat the headache disorder in the subject.

The subject invention also provides a method of treating epilepsy in a subject in need of such treatment comprising administering to the subject a therapeutically effective dose of any of the solid dosage forms or tablets of the invention in order to thereby treat epilepsy in the subject.

The subject invention also provides a method of controlling seizures in a subject suffering from epilepsy comprising administering to the subject a therapeutically effective dose of any of the solid dosage forms or tablets of the invention in order to thereby control the seizures in the subject.

The subject invention also provides a method of treating pain in a subject in need of such treatment comprising administering to the subject a therapeutically effective dose of any of the solid dosage forms or tablets of the invention in order to thereby treat pain in the subject.

The subject invention also provides a method of pain prophylaxis in a subject in need of such treatment comprising administering to the subject a prophylactic dose of any of the solid dosage forms or tablets of the invention in order to thereby effect pain prophylaxis in the subject.

The subject invention also provides a method of treating mania in bipolar disorder in a subject in need of such treatment comprising administering to the subject a therapeutically effective dose of any of the solid dosage forms or tablets of the invention in order to thereby treat mania in bipolar disorder in the subject.

The subject invention also provides a method of attenuating bipolar mood swings in a subject suffering from bipolar disorder comprising administering to the subject a therapeutically effective dose of any of the solid dosage forms or tablets of the invention in order to thereby attenuate the bipolar mood swings in the subject.

The subject invention also provides a process for preparing the above solid dosage form, comprising the steps of:

a) admixing predetermined amounts of

(i) an active ingredient selected from the group consisting of valproic sodium acid, a pharmaceutically acceptable salt or ester of valproic acid, divalproex sodium, valpromide and a compound having the structure:

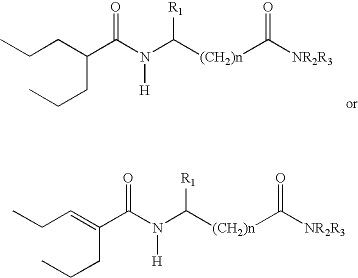

wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and

(ii) a binder;

b) admixing the uniform mixture of step a) with a predetermined amount of a hydroxypropylmethyl cellulose; and

c) compressing the mixture of step b) to form the tablet.

In one embodiment of the process, step b) further comprises admixing the uniform mixture with a predetermined amount of a different hydroxypropylmethyl cellulose.

In another embodiment, step b) further comprises admixing the uniform mixture with predetermined amounts of a filler, a lubricant and a flow agent.

In another embodiment, the flow agent comprises colloidal fumed silica.

In another embodiment, the filler comprises microcrystalline cellulose, anhydrous dicalcium phosphate, lactose or a combination of two or more of the foregoing.

In another embodiment, the filler comprises lactose.

In another embodiment, the lubricant comprises magnesium stearate or sodium stearyl fumarate or a combination thereof.

In another embodiment, the lubricant comprises magnesium stearate.

In another embodiment,

each hydroxypropylmethyl cellulose of step b) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

In another embodiment, at least 90% of the hydroxypropylmethyl cellulose passes through a No. 100 US standard sieve.

In another embodiment,

the first hydroxypropylmethyl cellulose has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

the second hydroxypropylmethyl cellulose has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

The subject invention also provides a process for preparing the above hard compressed tablet comprising the steps of:

a) admixing predetermined amounts of N-(2-Propylpentanoyl)glycinamide, hydroxypropylmethyl cellulose, and a different hydroxypropylmethyl cellulose; and

b) compressing the mixture of step a) to form the hard compressed tablet.

In one embodiment,

each hydroxypropylmethyl cellulose of step a) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

In another embodiment, at least 90% of the hydroxypropylmethyl cellulose passes through a No. 100 US standard sieve.

In another embodiment,

the hydroxypropylmethyl cellulose has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

the different hydroxypropylmethyl cellulose has an apparent viscosity of 6,138-9,030 millipascal-seconds (nominal value 7382 mPa.s) by rotation and 11,250-21,000 cP (nominal value 15,000 cp) by Ubbelhode at a concentration of 1% by weight in water at 20° C.

In another embodiment, step a) further comprises admixing predetermined amounts of a filler, lubricant and flow agent as additional components.

In another embodiment,

the filler comprises microcrystalline cellulose, anhydrous dicalcium phosphate, lactose or a combination of two or more of the foregoing;

the lubricant comprises sodium stearyl fumarate; and

the flow agent comprises colloidal fumed silica.

The subject invention also provides a process for preparing the above composition in granulate form, comprising granulating a predetermined amount of valproic sodium acid, a pharmaceutically acceptable salt or ester of valproic acid, divalproex sodium, valpromide or a compound having the structure:

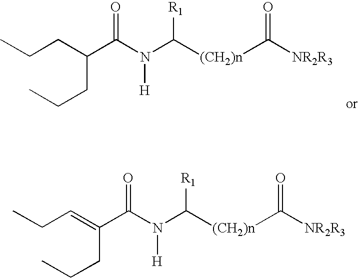

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, and a predetermined amount of hydroxypropyl cellulose to form the composition in granulate form.

The subject invention also provides a process for preparing a sustained release tablet comprising the steps of:

a) admixing the above granules with predetermined amounts of a hydroxypropylmethyl cellulose; and

b) compressing the mixture of step a) to form the tablet.

In another embodiment, step a) further comprises admixing the granules with a predetermined amount of each of a different hydroxypropylmethyl cellulose, a filler, a lubricant and a flow agent.

In another embodiment, the flow agent comprises colloidal fumed silica.

In another embodiment, the filler comprises microcrystalline cellulose, anhydrous dicalcium phosphate, lactose or a combination of two or more of the foregoing.

In another embodiment, the filler is lactose.

In another embodiment, the lubricant comprises magnesium stearate or sodium stearyl fumarate or a combination thereof.

In another embodiment, the lubricant comprises magnesium stearate.

In another embodiment, the process comprises the steps of:

a) admixing the granules with predetermined amounts of hydroxypropyl methyl cellulose having an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C., and hydroxypropyl methyl cellulose having an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and

b) compressing the mixture of step a) to form the tablet.

In another embodiment, step a) further comprises admixing the granules with predetermined amounts of a flow agent, a filler, and a lubricant.

In another embodiment, the process comprises the steps of

a) admixing the granules with

a predetermined amount of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C. which results in tablets containing 150 mg/tablet;

a predetermined amount of hydroxypropyl methyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C. which results in tablets containing 60 mg/tablet;

a predetermined amount of lactose which results in tablets containing 20 mg/tablet;

a predetermined amount of magnesium stearate which results in tablets containing 4.5 mg/tablet; and

a predetermined amount of a colloidal fumed silica which results in tablets containing 1 mg/tablet; and

b) compressing the mixture of step a) to form the tablet.

The subject invention also provides the use of an active ingredient selected from the group consisting of valproic sodium acid, a pharmaceutically acceptable salt or ester of valproic acid, divalproex sodium, valpromide and a compound having the structure:

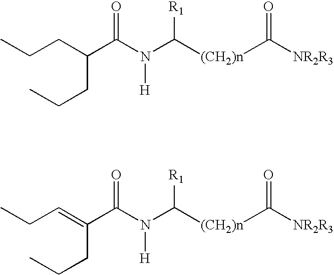

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in treating a headache disorder in a subject.

The subject invention also provides the use of an active ingredient selected from the group consisting of valproic sodium acid, a pharmaceutically acceptable salt or ester of valproic acid, divalproex sodium, valpromide and a compound having the structure:

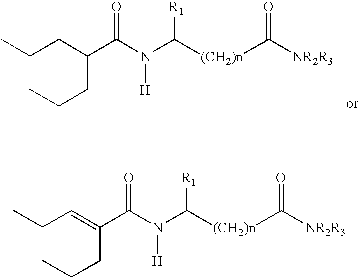

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in treating neuropathic pain in a subject.

The subject invention also provides the use of an active ingredient selected from the group consisting of valproic sodium acid, a pharmaceutically acceptable salt or ester of valproic acid, divalproex sodium, valpromide and a compound having the structure:

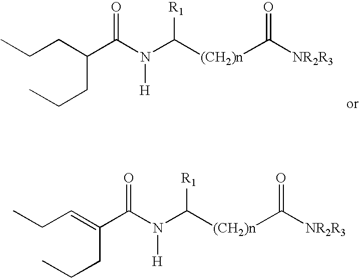

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage from or tablet of the invention for use in treating epilepsy in a subject.

The subject invention also provides the use of an active ingredient selected from the group consisting of valproic sodium acid, a pharmaceutically acceptable salt or ester of valproic acid, divalproex sodium, valpromide and a compound having the structure:

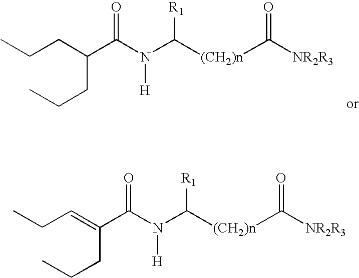

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid release dosage form or tablet of the invention for use in controlling seizures in a subject suffering from epilepsy.

The subject invention also provides the use of an active ingredient selected from the group consisting of valproic sodium acid, a pharmaceutically acceptable salt or ester of valproic acid, divalproex sodium, valpromide and a compound having the structure:

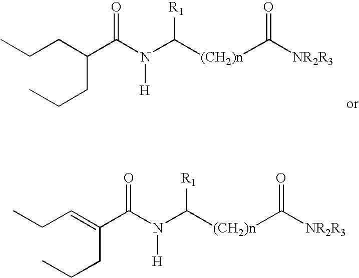

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in treating mania in bipolar disorder in a subject.

The subject invention also provides the use of an active ingredient selected from the group consisting of valproic sodium acid, a pharmaceutically acceptable salt or ester of valproic acid, divalproex sodium, valpromide and a compound having the structure:

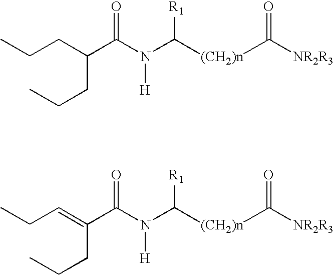

or

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in attenuating bipolar mood swings in a subject suffering from bipolar mood disorder.

The subject invention also provides the use of an active ingredient selected from the group consisting of valproic sodium acid, a pharmaceutically acceptable salt or ester of valproic acid, divalproex sodium, valpromide and a compound having the structure:

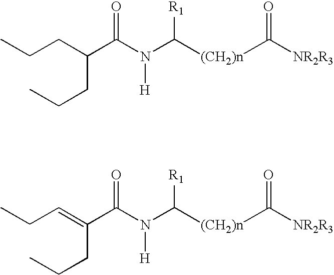

or

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in treating pain in a subject.

The subject invention also provides the use of an active ingredient selected from the group consisting of valproic sodium acid, a pharmaceutically acceptable salt or ester of valproic acid, divalproex sodium, valpromide and a compound having the structure:

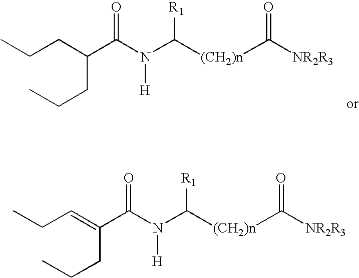

wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form or tablet of the invention for use in effecting pain prophylaxis in a subject.

The subject invention also provides the sustained release solid dosage form or tablet for use in treating a headache disorder in a subject.

The subject invention also provides the sustained release solid dosage form or tablet for use in treating neuropathic pain in a subject.

The subject invention also provides the sustained release solid dosage form or tablet for use in treating epilepsy in a subject.

The subject invention also provides the sustained release solid dosage form or tablet for use in controlling seizures in a subject suffering from epilepsy.

The subject invention also provides the sustained release solid dosage form or tablet for use in treating mania in bipolar disorder in a subject.

The subject invention also provides the sustained release solid dosage form or tablet for use in attenuating bipolar mood swings in a subject suffering from bipolar disorder.

The subject invention also provides the sustained release solid dosage form or tablet for use in treating pain in a subject.

The subject invention also provides the sustained release solid dosage form or tablet for use in effecting pain prophylaxis in a subject.

The subject invention also provides a controlled release oral unit dose composition comprising N-(2-propylpentanoyl) glycinamide and at least one pharmaceutically acceptable carrier, wherein the composition when orally ingested by a human subject, induces a peak blood plasma level of N-(2-propylpentanoyl)glycinamide between 4 and 24 hours after ingestion of a single oral unit dose.

In one embodiment, the composition when orally ingested by a human subject, induces a peak blood plasma level of N-(2-propylpentanoyl)glycinamide between 4 and 12 hours after ingestion of a single oral unit dose.

In a further embodiment, the composition, when orally ingested by a human subject, induces a peak blood plasma level of N-(2-propylpentanoyl)glycinamide between 6 and 12 hours after ingestion of a single oral unit dose.

In a further embodiment, the composition, when orally ingested by a human subject, induces a peak blood plasma level of N-(2-propylpentanoyl)glycinamide between 6 and 8 hours after ingestion of a single oral unit dose.

In a further embodiment of the above controlled release oral dose compositions, the peak blood plasma level of N-(2-propylpentanoyl)glycinamide is from 0.5 micrograms/ml to 16 micrograms/ml per a 1000 mg dose of N-(2-propylpentanoyl) glycinamide in the composition.

In another embodiment, the composition, when orally ingested by a human subject, induces a peak blood plasma level of N-(2-propylpentanoyl)glycine in the human subject from 0.5 μg/mL to 1.7 μg/mL per a 1000 mg dose of N-(2-propylpentanoyl) glycinamide in the composition.

The subject invention also provides a controlled release oral dose composition comprising N-(2-propylpentanoyl)glycinamide 43- and a pharmaceutically acceptable carrier, wherein the composition when orally ingested by a human subject, induces a peak blood plasma level of N-(2-propylpentanoyl)glycinamide of 0.5 μg/mL to 16 μg/mL per a 1000 mg dose in the composition.

The subject invention also provides a controlled release oral dose composition comprising N-(2-propylpentanoyl)glycinamide and a pharmaceutically acceptable carrier, wherein the composition when orally ingested by a human subject, induces a peak blood plasma level of N-(2-propylpentanoyl)glycine of 0.5 μg/mL to 1.7 μg/mL per a 1000 mg dose of N-(2-propylpentanoyl) glycinamide in the composition.

The subject invention also provides a method of inducing in a human subject a peak blood plasma level of N-(2-propylpentanoyl)glycinamide between 4 and 24 hours after administration of N-(2-propylpentanoyl)glycinamide, comprising administering to the human subject a controlled release oral unit dose composition comprising N-(2-propylpentanoyl) glycinamide and at least one pharmaceutically acceptable carrier, which composition induces a peak blood plasma level of N-(2-propylpentanoyl)glycinamide between 4 and 24 hours after administration of a single oral unit dose.

In one embodiment, the peak blood plasma level of N-(2-propylpentanoyl)glycinamide occurs between 4 and 12 hours after administration.

In another embodiment, the peak blood plasma level of N-(2-propylpentanoyl)glycinamide is 0.5 μg/mL to 16 μg/mL per 1000 mg dose of N-(2-propylpentanoyl)glycinamide in the composition.

In one embodiment of any of the above methods, the administeration to the human subject of a controlled release oral unit dose composition comprising N-(2-propylpentanoyl) glycinamide and at least one pharmaceutically acceptable carrier induces a peak blood plasma level of N-(2-propylpentanoyl)glycine in the human subject from 0.5 μg/mL to 1.7 μg/mL upon administration of a single 1000 mg dose of N-(2-propylpentanoyl)glycinamide.

In another embodiment, the controlled release oral dose composition is any of the solid dosage forms or the tablets described above.

In another embodiment of the invention, the process for manufacturing the sustained release formulation of N-(2-Propylpentanoyl)glycinamide comprises:

1. Preparing a granulate of N-(2-Propylpentanoyl)glycinamide

2. Mixing the granulate of step 1 with excipients

3. Compressing the mixture of step 2 to form a sustained release tablet of N-(2-Propylpentanoyl)glycinamide

In another embodiment, the process for manufacturing the sustained release formulation of N-(2-Propylpentanoyl)glycinamide comprises:

1. Mixing the active material with a carrier and other excipients

2. Direct compression of the mixture of step 1.

In another embodiment, the process for manufacturing the sustained release formulation of N-(2-Propylpentanoyl)glycinamide comprised:

1. Mixing N-(2-Propylpentanoyl)glycinamide with a carrier and other excipients

2. Compression of the mixture of step 1 into tablets

3. Preparing slugs of the tablets of step 2

4. Filling into capsules the slugs of step 3

In another embodiment, the process for manufacturing the sustained release formulation of N-(2-Propylpentanoyl)glycinamide comprised:

1. Mixing N-(2-Propylpentanoyl)glycinamide with a carrier and other excipients

2. Compression of the mixture of step 1 into tablets

3. Preparing slugs of the tablets of step 2

4. Dispersing the slugs of step 3 in suspension

“Slugs” are granulates manufactured via a dry granulation process that involves milling the tablets into small particles.

The present invention provides a sustained release pharmaceutical composition comprising the active-material N-(2-Propylpentanoyl)glycinamide.

The subject invention also provides an oral dosage of N-(2-Propylpentanoyl)glycinamide sustained release form.

As used herein, “US Standard Sieve No. 40” refers to a sieve having a specified sieve opening of 0.0165 inches and a specified wire diameter of 0.0098 inches.

As used herein, “US Standard Sieve No. 100” refers to a sieve having a specified sieve opening of 0.0059 inches and a specified wire diameter of 0.0040 inches.

As used herein, the phrase “controlled release” dosage forms refer to dosage forms which are formulated to release the drug slowly over a prolonged period of time. These dosage forms are also referred to as “sustained-release” or “prolonged release” dosage forms (Remington: The Science and Practice of Pharmacy, 20^{th} ed. P. 859). However, the term “controlled release” also includes enterically coated tablets while the term “sustained release” does not.

As used herein, the term “compressed tablets” refers to tablets which formed by a press tableting machine which applies a compression force of between about 2000 lb (about 8.9×10³ Newtons) and about 10,000 lb (4.45×10⁴ Newtons).

As used herein, the term “hard compressed tablets” refers to tablets which remain unchanged under compression forces ranging from about 2000 lb (1.3×10⁴ Newtons) to about 10,000 lb (4.45×10⁴ Newtons). The term “hard compressed tablets” does not include within its scope any granulate which does not itself meet the test for hardness described above.

There are several in vitro mechanisms by which the N-(2-Propylpentanoyl)glycinamide can be released. One such mechanism is sustained release in matrix tablets. The main principle of this mechanism is that the water partially hydrates the outer layers of the tablet to form a gel layer. Throughout the life of the ingested tablet, the rate of drug diffusion and of the wet gel and the rate of the tablet erosion control the overall dissolution rate and drug availability.

This matrix can be obtained by direct compression or by initial granulation, which granules are then compressed into the matrix system. In monolithic matrix systems, the drug is homogeneously dispersed throughout a polymer mass of other carrier material.

Release characteristics depend on the geometry of the system, the nature of the polymer and other excipients, solubility and the processing methods.

N-(2-Propylpentanoyl)glycinamide is difficult to work with due to its “lamination and compression” characteristics. To alleviate the problem of lamination, the subject invention employs a filler and hydroxypropylmethyl cellose as a carrier which improve the compressing characteristics while simultaneously slowing down the release profile.

In a preferred embodiment, the carrier is Methocel k100 LV, and the filler is lactose.

As described more fully in the examples which follow, in order to develop a prototype with a slower dissolution profile, the concentration of the carrier (e.g. methocel) was increased until any further increase gave no effect on the resulting dissolution profile. At this point, the polymer had achieved the maximum sustained action.

In order to further improve the dissolution profile, a second molecular weight grade of methocel was added to the formulation. While the first grade of Methocel improved the compression properties and achieved a maximum sustained action, the second grade detracted from the physical characteristics of the tablet but improved the sustained-release action. However, by combining these two different molecular weight grades of methocel in the correct proportions, the dissolution rate was decreased and the tablets were made with the desired physical characteristics.

Thus, the subject invention provides a sustained release formulation of N-(2-Propylpentanoyl)glycinamide which contains two different grades of Methocel combined in the correct proportions to achieve the desired dissolution profile and the desired compressibility characteristics.

In the case of compression tablets, the excipients give the desired flow of granules, and uniform compressibility into tablets.

The pharmaceutical excipients include fillers, flow agents, disintegrants and lubricants.

Most multiparticulate systems are delivered in the form of solid dosage. However, for some patients, it is desirable to use extended release dosage forms in liquid form. The multiparticulate system can be a redispersable dosage form, or a liquid suspension.

Non-limiting examples of a filler used in the subject invention (used for example for weight adjustment and for better compression) are corn starch, lactose, glucose, various natural gums, methylcellulose, carboxymethylcellulose, microcrystalline cellulose (e.g. Avicel® PH101 or 102 (FMC Corporation, Philadelphia, Pa.)), calcium phosphate, calcium carbonate, calcium sulfate kaolin, sodium chloride, powdered cellulose, sucrose, mannitol and starch. In a preferred embodiment, the excipient useful as a filler comprises a microcrystalline cellulose.

Non-limiting examples of a carrier (extended release agent) used in the subject invention (used for example for the controlled release) are cellulose acetate, glyceryl monostearate, zein, microcrystalline wax, hydroxymethylcellulose, hydroxyethylcellulose, hydroxypropylmethylcellulose, hydroxypropylcellulose (e.g., Klucel®), carboxyvinyl polymers, polyvinyl alcohols, glucans, scleroglucans, chitosans, mannans, galactomannans, amylose, alginic acid and salts and derivatives thereof, acrylates, methacrylates, acrylic/methacrylic copolymers, polyanhydrides, polyaminoacids, methyl vinyl ethers/maleic anhydride copolymers, carboxymethylcellulose and derivatives thereof, ethylcellulose, methylcellulose and cellulose derivatives in general, modified starch and polyesters, polyethylene oxide.

In an embodiment, the excipient used as a carrier comprises a hydroxypropylmethylcellulose. In another embodiment, the hydroxypropylmethylcellulose has an average molecular weight between about 10 kDa and about 1500 kDa. In a further embodiment, the hydroxypropylmethylcellulose has 19%-24% methoxyl substituent and 7%-12% hydroxylproproxyl substituent. In an added embodiment, the hydroxypropylmethyl cellulose has a pH of 5.5-8.0 in a 1% solution. In an added embodiment, the hydroxypropylmethylcellulose has a particle size distribution such that about 100% of the hydroxypropylmethylcellulose passes through a 30 mesh screen. In one embodiment, the hydroxypropylmethylcellulose has a particle size distribution such that about 99% of the hydroxypropylmethylcellulose passes through a 40 mesh screen. In yet another embodiment, the hydroxypropylmethylcellulose has a particle size distribution such that 55%-95% of the hydroxypropylmethylcellulose passes through a 100 mesh screen. In yet another embodiment, the hydroxypropylmethylcellulose has a particle size distribution such that 90% of the hydroxypropylmethylcellulose passes through a 100 mesh screen. In a further embodiment, the hydroxypropylmethylcellulose has a particle size distribution such that 65%-85% of the hydroxypropylmethylcellulose passes through a 100 mesh screen. In an additional embodiment, the hydroxypropylmethylcellulose has a particle size distribution such that about 80% of the hydroxypropylmethylcellulose passes through a 100 mesh screen. In a further embodiment, the hydroxypropylmethylcellulose has a particle size distribution such that about 90% of the hydroxypropylmethylcellulose passes through a 100 mesh screen. In a further embodiment, the hydroxypropylmethylcellulose is a Methocel® polymer (Colorcon, West Point, Pa.), such as Methocel® K100 Premium LV EP or LV LH EP alone or in combination, or Methocel® K15M EP or CR EP.

Non-limiting examples of a binding agent used in the subject invention (used for example for the granulate) are alginic acid, acia, carbomer, carboxymethylcellulose sodium, dextrin, ethylcellulose, gelatin, guar gum, hydrogenated vegetable oil, hydroxyethylcellulose, hydroxypropylcellulose (e.g., Klucel®, Aqualon Division, Hercules Incorporated, Wilmington, Del.), hydroxypropylmethylcellulose, liquid glucose, magnesium aluminum silicate, maldodextrin, methylcellulose, polymethacrylates, povidone, pregelatinized starch, sodium alginate, starch, and zein. In a preferred embodiment, the excipient used as a binding agent comprises a hydroxypropylcellulose.

In one embodiment, the excipient used as a binder is hydroxypropyl cellulose. In one embodiment, the hydroxypropyl cellulose has a particle size distribution such that about 85% of the hydroxypropyl cellulose passes through a 30 mesh screen.

In another embodiment, the hydroxypropyl cellulose has a particle size distribution such that about 99% of the hydroxypropyl cellulose passes through a 20 mesh screen. In another embodiment, the hydroxypropyl cellulosehas a pH of 5.0-7.5 in water solution. In one embodiment, the hydroxypropyl cellulose has an average molecular weight of 1,150,000. In one embodiment, the hydroxypropyl cellulose has an average molecular weight of 850,000. In one embodiment, the hydroxypropyl cellulose has an average molecular weight of 370,000. In one embodiment, the hydroxypropyl cellulose has an average molecular weight of 140,000. In one embodiment, the hydroxypropyl cellulose has an average molecular weight of 95,000. In one embodiment, the hydroxypropyl cellulose has an average molecular weight of 80,000. In one embodiment, the hydroxypropyl cellulose has a viscosity of 1,500-3,000 cps at a concentration of 1% by weight in water at 25° C. In one embodiment, the hydroxypropyl cellulose has a viscosity of 4,000-6,500 cps at a concentration of 2% by weight in water at 25° C. In one embodiment, the hydroxypropyl cellulose has a viscosity of 150-400 cps at a concentration of 2% by weight in water at 25° C. In one embodiment, the hydroxypropyl cellulose has a viscosity of 150-400 cps at a concentration of 5% by weight in water at 25° C. In one embodiment, the hydroxypropyl cellulose has a viscosity of 75-150 cps at a concentration of 5% by weight in water at 25° C. In one embodiment, the hydroxypropyl cellulose has a viscosity of 200-600 cps at a concentration of 10% by weight in water at 25° C. In one embodiment, the hydroxypropyl cellulose has a viscosity of 75-150 cps at a concentration of 5% by weight in water at 25° C. In one embodiment, the hydroxypropyl cellulose has a viscosity of 300-600 cps at a concentration of 10% by weight in water at 25° C.

In one embodiment, the excipient used as a filler is a microcrystalline cellulose. In an added embodiment, the microcrystalline cellulose has an average particle size between about 50 and about 90 microns.

Non-limiting examples of a flow agent used in the subject invention are micron-sized silica powders. A non-limiting example of a flow agent used in the subject invention (used for better flow of the mix for compression) is colloidal silicon dioxide or Syloid®.

Non-limiting examples of a lubricant used in the subject invention (used for example for better compression properties) are talc, sodium stearyl fumarate, magnesium stearate, calcium stearate, hydrogenated castor oil, hydrogenated soybean oil and polyethylene glycol (PEG) or combinations thereof.

Details of general formulation procedures and information on additional excipients may be found in Remington: The Science and Practice of Pharmacy, 20^{th} Edition.

This invention will be better understood from the Experimental Details which follow.

### EXPERIMENTAL DETAILS

### Example 1

### Manufacture of N-(2-Propylpentanoyl)glycinamide Sustained Release (SR) Tablets

Granules of N-(2-Propylpentanoyl)glycinamide:

N-(2-n-Propylpentanoyl)glycinamide was granulated with a binder solution and with several excipients.

TABLE 1
Composition of the granules

| Excipient | Use | Mg/tablet |
|---|---|---|
| N-(2-Propylpentanoyl) | Active material | 500 |
| glycinamide | | |
| Microcrystalline Cellulose | Filler | 100 |
| Hydroxypropyl cellulose | Binder | 50 |
| Total | | 650 |

The tablets were then prepared by mixing the granulate with a carrier/carriers and several excipients (table 2).

TABLE 2
Composition of the sustained release tablets

| | | Mg/Tablet | | |
|---|---|---|---|---|
| Excipient | Use | A | B | C |
| N-(2-Propylpentanoyl) | | 650 | 650 | 650 |
| glycinamide Granulate | | | | |
| Aerosil | Flow-agent | 1.0 | 1.0 | |
| Lactose | Filler | 80 | 20 | 145 |
| Hydroxypropyl Methyl | Carrier | — | 60 | |
| Cellulose | | | | |
| (Methocel K15M) | | | | |
| Hydroxypropyl Methyl | Carrier | 150 | 150 | |
| Cellulose | | | | |
| (Methocel K 100LV) | | | | |
| Magnesium Stearate | Lubricant | 4.5 | 4.5 | 6 |
| Crosscarmelose Sodium | Disintegrant | | | 50 |

Each formulation was tested in a dissolution test using 900 ml purified water, 37° C., in US Pharmacopoeia (USP), versus the immediate release formulation (Formulation C).

TABLE 3
Dissolution of N-(2-Propylpentanoyl)glycinamide SR tablets

| | Formula | | |
|---|---|---|---|
| Time (h) | A | B | C |
| | % Dissolution | | |
| 0.5 | 7 | 4 | 100 |
| 2 | 34 | 15 | |
| 4 | 66 | 32 | |
| 6 | 88 | 48 | |
| 10 | 102 | 75 | |
| 12 | | 86 | |
| 14 | | 96 | |
| 16 | | 102 | |

As can be seen two different prototypes (A, B) of N-(2-Propylpentanoyl)glycinamide sustained release characteristics were observed.

### Example 2

### Effect of Carrier on Dissolution Rate

Each of the following formulations contained different carriers in order to determine the effect of the carrier on the dissolution rate.

TABLE 4
Variations In the carriers

| | | Formula | | | | |
|---|---|---|---|---|---|---|
| | | D | E | F | G | H |
| | | Methocel | Klucel | Carbopol | Methocel | Methocel |
| Excipient | Use | K100M* | HF* | 974p* | K100LV* | K15M* |
| | | Mg/Tablet | | | | |
| N-(2- | | 650 | 650 | 650 | 650 | 650 |
| Propylpentanoyl) | | | | | | |
| glycinamide | | | | | | |
| Granulate | | | | | | |
| Aerosil | Flow- | 16.5 | 16.5 | 16.5 | 16.5 | 16.5 |
| | agent | | | | | |
| Lactose | Filler | 80 | 80 | 80 | 80 | 80 |
| *Carrier | *Carrier | 120 | 120 | 120 | 100 | 120 |
| Magnesium | Lubricant | 4.5 | 4.5 | 4.5 | 4.5 | 4.5 |
| Stearate | | | | | | |

Each formulation was tested in a dissolution test using 900 ml purified water 37° C., in US Pharmacopoeia (USP). The dissolution profile was found to be dependent upon the type of the carrier.

TABLE 5
Dissolution of tablets D-H

| | Formula | | | | |
|---|---|---|---|---|---|
| | D | E | F | G | H |
| | Methocel | Klucel | Carbopol | Methocel | Methocel |
| Time (h) | K100M | HF | 974p | K100LV | K15M |
| | % Dissolution | | | | |
| 0.5 | 2 | 5 | 7 | 12 | 7 |
| 1 | 4 | 8 | 16 | 26 | 15 |
| 2 | 8 | 12 | 26 | 52 | 32 |
| 3 | 10 | 16 | 30 | 71 | 47 |
| 4 | 13 | 19 | 33 | 86 | 61 |
| 6 | 17 | 25 | 39 | 106 | 83 |
| 8 | 21 | 30 | 45 | | 100 |
| 12 | 29 | 40 | 54 | | |

Due to their resulting dissolution profile of 6-8 hours, Methocel K100LV and/or Methocel K15M were selected as suitable carriers.

### Example 3

### Effect of the Amount of Carrier on Dissolution Rate

In order to determine the effect of the amount of the carrier on the dissolution rate, formulations were tested while varying the amount of Methocel K100 LV and/or Methocel K15M.

TABLE 6
Variation in the amount of the carrier (Methocel K100 LV)

| | | Formula | | |
|---|---|---|---|---|
| Excipient | Use | I | J | K |
| | | Mg/Tablet | | |
| N-(2- | | 650 | 650 | 650 |
| Propylpentanoyl) | | | | |
| glycinamide | | | | |
| Granulate | | | | |
| Aerosil | Flow-agent | 1.0 | 1.0 | 1.0 |
| Lactose | Filler | 80 | 80 | 60 |
| Hydroxypropyl | Carrier | 100 | 150 | 170 |
| Methyl Cellulose | | | | |
| (Methocel K100 | | | | |
| LV) | | | | |
| Magnesium | Lubricant | 4.5 | 4.5 | 4.5 |
| Stearate | | | | |

Each formulation was then tested in a dissolution test using 900 ml purified water, 37° C., in US Pharmacopoeia (USP).

TABLE 7
Dissolution of formulations I-K

| | Formula | | |
|---|---|---|---|
| Time (h) | I | J | K |
| | % Dissolution | | |
| 0.5 | 15 | 11 | 8 |
| 1 | 28 | 20 | 12 |
| 2 | 49 | 39 | 35 |
| 3 | 64 | 54 | 51 |
| 4 | 76 | 68 | 65 |
| 6 | 94 | 87 | 87 |
| 8 | 104 | 98 | 102 |
| 12 | 105 | 105 | 110 |

The results showed that the dissolution profile was dependent upon the amount of the carrier (Methocel K100LV). Increasing the concentration of the polymer in the matrix system increases the viscosity of the outer layer gel which forms and leads to a more delayed release of the drug product. However, when increasing the amount of carrier from formulation J to formulation K the effect on the endpoint of the dissolution was less significant than the change observed when changing from formulation I to J. Thus, formulation J achieves the maximum sustained action for this polymer.

The same procedure was followed in order to determine the effect of the amount of Methocel® K15M on the dissolution profile.

TABLE 8
Variation in the amount of the carrier (Methocel K15M)

| | | Formula | | |
|---|---|---|---|---|
| Excipient | Use | L | M | N |
| | | Mg/Tablet | | |
| N-(2- | | 650 | 650 | 650 |
| Propylpentanoyl) | | | | |
| glycinamide | | | | |
| Granulate | | | | |
| Aerosil | Flow-agent | 1.0 | 1.0 | 1.0 |
| Lactose | Filler | 80 | 80 | 60 |
| Hydroxypropyl | Carrier | 80 | 100 | 150 |
| Methyl Cellulose | | | | |
| (Methocel K15M) | | | | |
| Magnesium | Lubricant | 4.5 | 4.5 | 4.5 |
| Stearate | | | | |

Each formulation was tested in a dissolution test using 900 ml purified water, 37° C., in US Pharmacopoeia (USP).

TABLE 9
Dissolution of formulations L-N

| | Formula | | |
|---|---|---|---|
| Time (h) | L | M | N |
| | % Dissolution | | |
| 0.5 | 9 | 6 | 4 |
| 1 | 13 | 10 | 7 |
| 2 | 21 | 17 | 12 |
| 3 | 29 | 23 | 17 |
| 4 | 37 | 30 | 22 |
| 6 | 50 | 43 | 31 |
| 8 | 63 | 55 | 40 |
| 10 | 75 | 66 | 49 |
| 12 | 84 | 75 | 57 |
| 14 | | 84 | |
| 16 | | 90 | 72 |
| 18 | | | 76 |

The results showed that the dissolution profile was dependent upon the amount of the carrier (Methocel® K15M) and that increasing the concentration of this polymer in the matrix system delays the release of the drug product. More polymer in the matrix leads to more polymer on the tablet surface. Hence, wetting is more easily achieved and gel formation is accelerated. However, formulation N suffered from poor compressibility characteristics.

The use of Methocel K15M as a carrier was found to slow the dissolution profile. However, it also yielded tablets with poor compressibility properties. Other alternatives were therefore investigated in order to produce tablets with good compressibility properties as well as slow dissolution profiles.

### Example 4

### Effect of Time From Production on Dissolution Rate

TABLE 10
Time effect of production on the dissolution profile

Mg/Tablet

| Excipient | Use | O | P |
|---|---|---|---|
| N-(2- | | 650 | 650 |
| Propylpentanoyl) | | | |
| glycinamide | | | |
| Granulate | | | |
| Aerosil | Flow-agent | 1.0 | 1.0 |
| Lactose | Filler | 80 | 80 |
| Hydroxypropyl | Carrier | 150 | 150 |
| Methyl Cellulose | | | |
| (Methocel K100LV) | | | |
| Magnesium | Lubricant | 4.5 | 4.5 |
| Stearate | | | |

O: The tablets were kept in uncontrolled conditions for two years.
P: The same formulation was compressed anew.

The formulations were then checked for dissolution profile.

TABLE 11
Dissolution of tablets O-P

Formula

| | | |
|---|---|---|
| Time (h) | O | P |

% Dissolution

| | | |
|---|---|---|
| 0.5 | 9 | 11 |
| 1 | 20 | 20 |
| 2 | 39 | 39 |
| 3 | 55 | 54 |
| 4 | 67 | 68 |
| 6 | 85 | 87 |
| 8 | 95 | 98 |
| 12 | 101 | 105 |

The results indicated that the formulations utilized were extremely stable.

### Example 5

### Effect of Combining Methocel Carriers on Dissolution

TABLE 12
Effect of combining different amounts of of Methocel carriers.

Formula

| | | | |
|---|---|---|---|
| Excipient | Use | Q | S |

Mg/Tablet

| | | | |
|---|---|---|---|
| N-(2- | | 650 | 650 |
| Propylpentanoyl) | | | |
| glycinamide | | | |
| Granulate | | | |
| Aerosil | Flow- | 1.0 | 1.0 |
| | agent | | |
| Lactose | Filler | 40 | 40 |
| Carrier (Methocel | Carrier | 150 | 150 |
| K100LV) | | | |
| Carrier (Methocel | Carrier | 40 | 75 |
| K15M) | | | |
| Magnesium | Lubricant | 4.5 | 4.5 |
| Stearate | | | |

Each formulation was tested in a dissolution test using 900 ml purified water, 37° C., in US Pharmacopoeia (USP).

TABLE 13
Dissolution proflie of tablets Q and S

Formula

| | |
|---|---|
| Q | S |

| | |
|---|---|
| Time (h) | % Dissolution |

| | | |
|---|---|---|
| 0.5 | 6 | 5 |
| 2 | 23 | 18 |
| 4 | 46 | 34 |
| 6 | 65 | 49 |
| 8 | 80 | |
| 10 | 92 | 73 |
| 12 | 100 | 87 |
| 14 | 105 | 91 |
| 16 | | 94 |

As illustrated by the above results, increasing the amount of Methocel K15M relative to Methocel K100 LV improved and decreased the dissolution rate.

### Example 6

### Effect of Lubricant Type and Amount on Dissolution Rate

TABLE 14
Effect of Lubricant type and amount

| | | Formula | | | |
|---|---|---|---|---|---|
| Excipient | Use | V | W | X | Y |
| | | Mg/Tablet | | | |
| N-(2-n- | | 650 | 650 | 650 | 650 |
| Propylpentanoyl) | | | | | |
| glycinamide | | | | | |
| Granulate | | | | | |
| Aerosil | Flow-agent | 1.0 | 1.0 | 1.0 | 1.0 |
| Lactose | Filler | 20 | 20 | 80 | 80 |
| Methocel K100LV | Carrier | 150 | 150 | 150 | 150 |
| Methocel K15M | Carrier | 60 | 60 | — | — |
| Magnesium Stearate | Lubricant | 4.5 | — | 4.5 | — |
| Sodium Stearyl | Lubricant | — | 9.0 | — | 9.0 |
| Fumarate (Pruv) | | | | | |

Each formulation was tested in a dissolution test using 900 ml purified water, 37° C., in US Pharmacopoeia (USP).

TABLE 15
Dissolution profile of tablets V-Y

Formula

| | | | |
|---|---|---|---|
| V | W | X | Y |

| | |
|---|---|
| Time (h) | % Dissolution |

| | | | | |
|---|---|---|---|---|
| 0.5 | 4 | 4 | 7 | 7 |
| 2 | 15 | 16 | 34 | 34 |
| 4 | 32 | 34 | 66 | 66 |
| 6 | 48 | 52 | 88 | 89 |
| 10 | 75 | 78 | 102 | 103 |
| 12 | 86 | 88 | | |
| 14 | 96 | 95 | | |
| 16 | 102 | 101 | | |
| 18 | | 105 | | |

As can be seen, no effect on dissolution profile was observed when changing the lubricant type or quantity. However, the physical comprising properties were improved when using Pruv instead of magnesium stearate.

### Example 7

### Effect of Apparatus Type on Dissolution Rate

TABLE 16
Influence of apparatus type

| | | Formulation | |
|---|---|---|---|
| | | A | B |
| Excipient | Use | mg/tablet | mg/tablet |
| N-(2- | Active | 650 | 650 |
| propylpentanoyl)glycinamide | | | |
| granulate | | | |
| Aerosil | Flow-agent | 1.0 | 1.0 |
| Lactose | Filler | 80 | 20 |
| Methocel K100LV | Carrier | 150 | 150 |
| Methocel K15M | Carrier | 0 | 60 |
| Magnesium Stearate | Lubricant | 4.5 | 4.5 |

The formulations were tested using dissolution tests using two different apparatuses using 900 mL purified water at 37° C., according to US Pharmacopoeia (USP). Apparatus 1 (basket apparatus) was maintained at 100 RPM. Apparatus 2 (paddle apparatus) was maintained at 75 RPM.

TABLE 17
Dissolution profile of tablets according to
Apparatus 1 and Apparatus 2

| | Formulation A | | Formulation B | |
|---|---|---|---|---|
| | Apparatus 1 | Apparatus 2 | Apparatus 1 | Apparatus 2 |
| Time (h) | % dissolution | | % dissolution | |
| 0.5 | 7 | 8 | 4 | 4 |
| 2 | 34 | 34 | 15 | 18 |
| 4 | 66 | 64 | 32 | 36 |
| 6 | 88 | 87 | 48 | 53 |
| 10 | 108 | 104 | 75 | 79 |
| 12 | | | 86 | 88 |
| 14 | | | 96 | 95 |
| 16 | | | 102 | 105 |

Results: The apparatus type used did not significantly the dissolution rate.

### Example 8

### Effect of Manufacturing Procedure on Dissolution

TABLE 18
Direct compression (DC, DC1, DC2,
DC3) versus wet granulation (W)

| | | Formula | | | | |
|---|---|---|---|---|---|---|
| Excipient | Use | W | DC | DC1 | DC2 | DC3 |
| | | mg/ tablet | | | | |
| N-(2- | Active | 650 | 500 | 750 | 750 | 750 |
| propylpentanoyl ) | | granulate | active | active | active | active |
| glycinamide | | | only | only | only | only |
| Aerosil (syloid) | Flow | 1.0 | 10 | 15 | 15 | 15 |
| | agent | | | | | |
| Lactose | Filler | 20 | 20 | | | |
| Methocel K100LV | Carrier | 150 | 150 | 150 | 150 | 150 |
| Methocel K15M | Carrier | 60 | 60 | | | |
| Methocel K15MCR | Carrier | | | 60 | 100 | 60 |
| Pruv | Lubricant | 9 | 10 | 15 | 15 | 20 |

The formulations were tested in standard dissolution tests using 900 ml purified water at 37° C. according to USP.

TABLE 19
Dissolution profile of tablets

| | Formula | | | | |
|---|---|---|---|---|---|
| Time (h) | W | DC | DC1 | DC2 | DC3 |
| | % dissolution | | | | |
| 0.5 | 4 | 7 | 13 | 8 | 12 |
| 2 | 16 | 18 | 27 | 18 | 27 |
| 4 | 34 | 32 | 44 | 31 | 43 |
| 6 | 52 | 44 | 58 | 42 | 59 |
| 10 | 78 | 67 | 71 | 53 | 72 |
| 12 | 88 | 77 | 83 | 63 | 85 |
| 14 | 95 | 85 | 94 | 73 | 96 |
| 16 | 101 | 91 | 100 | 81 | 101 |
| 18 | 105 | 95 | 102 | 88 | 103 |

These results show that although the active material is difficult to work with due to its unsatisfactory compression charateristics, direct compression technology and wet granulation technology both yielded tablets with a slow dissolution profile.

TABLE 20
Effect of lactose on the dissolution rate

| | | Mg/Tablet | | | | | |
|---|---|---|---|---|---|---|---|
| Excipient | Use | EE | FF | GG | HH | II | JJ |
| N-(2- | | 650 | 650 | 650 | 650 | 650 | 650 |
| Propylpentanoyl) | | | | | | | |
| glycinamide | | | | | | | |
| Granulate | | | | | | | |
| Aerosil | Flow- | 1.0 | 1.0 | 1.0 | 1.0 | 1.0 | 1.0 |
| | agent | | | | | | |
| Lactose | Filler | 40 | — | 40 | — | 80 | 40 |
| Methocel | Carrier | 150 | 150 | 150 | 150 | 100 | 100 |
| K100LV | | | | | | | |
| Methocel K15M | Carrier | 40 | 40 | 75 | 75 | — | — |
| Magnesium | Lubricant | 4.5 | 4.5 | 4.5 | 4.5 | 4.5 | 4.5 |
| Stearate | | | | | | | |

Each formulation was tested in a dissolution test using 900 ml purified water, 37° C., in US Pharmacopoeia (USP).

TABLE 21
Dissolution profile of tablets EE-JJ

Formula

| Time (h) | EE | FF | GG | HH | II | JJ |
|---|---|---|---|---|---|---|
| | % Dissolution | | | | | |
| 0.5 | 6 | 5 | 5 | 4 | 13 | 11 |
| 2 | 23 | 19 | 18 | 17 | 48 | 42 |
| 4 | 46 | 38 | 34 | 34 | 82 | 71 |
| 6 | 65 | 55 | 49 | 49 | 101 | 87 |
| 8 | 80 | 69 | | 65 | 106 | 96 |
| 10 | 92 | 81 | 73 | 73 | 106 | 100 |
| 12 | 100 | 91 | | 90 | 107 | 100 |
| 14 | 104 | 98 | 87 | 96 | | 100 |
| 16 | | 101 | 91 | 100 | | |

As can be seen, in formulations containing higher amounts of lactose, the dissolution rate was faster. However, the influence of the lactose decreased when the amount of the carrier increased and the influence of the carrier became more effective.

### Example 10

### Additional Testing on the Effect of Different Lubricants and pH on Dissolution Profile

TABLE 22
Formulations tested

| | | Mg/Tablet | | | |
|---|---|---|---|---|---|
| Excipient | Use | A | B | W | Y |
| N-(2-Propylpentanoyl) | | 650 | 650 | 650 | 650 |
| glycinamide Granulate | | | | | |
| Aerosil | Flow- | 1.0 | 1.0 | 1.0 | 1.0 |
| | agent | | | | |
| Lactose | Filler | 80 | 20 | 20 | 80 |
| Hydroxypropyl Methyl | Carrier | — | 60 | 60 | — |
| Cellulose (Methocel | | | | | |
| K15M) | | | | | |
| Hydroxypropyl Methyl | Carrier | 150 | 150 | 150 | 150 |
| Cellulose (Methocel | | | | | |
| K 100LV) | | | | | |
| Magnesium Stearate | Lubricant | 4.5 | 4.5 | — | — |
| Pruv | Lubricant | — | — | 9.0 | 9.0 |

TABLE 23
Dissolution profiles. Formula A with Magnesium Stearate

| | Basket | Paddle | Basket | Paddle | Paddle | Basket |
|---|---|---|---|---|---|---|
| | 100 | 75 | 100 rpm | 75 rpm | 75 rpm | 100 rpm |
| Intervals | rpm | rpm | 0.1 | Intestinal | Gastric | Phosphate |
| (min.) | Water | Water | N HCl | fluid | fluid | pH = 6.8 |
| 30 | 7 | 8 | 7 | 8 | 7 | 7 |
| 60 | 15 | 17 | 16 | 16 | 15 | 14 |
| 120 | 33 | 34 | 33 | 32 | 31 | 29 |
| 180 | 50 | 50 | 48 | 46 | 44 | 44 |
| 240 | 63 | 65 | 61 | 57 | 57 | 57 |
| 360 | 85 | 87 | 82 | 78 | 76 | 77 |
| 480 | 98 | 99 | 98 | 91 | 89 | 92 |
| 600 | 103 | | 102 | | | 101 |
| 720 | 103 | | 103 | | | 103 |

TABLE 24
Dissolution profiles. Formula B with Magnesium Stearate

| | Basket | Paddle | Basket | Paddle | Paddle | Basket |
|---|---|---|---|---|---|---|
| | 100 | 75 | 100 rpm | 75 rpm | 75 rpm | 100 rpm |
| Intervals | rpm | rpm | 0.1 | Intestinal | Gastric | Phosphat |
| (min.) | Water | Water | N HCl | fluid | fluid | pH = 6.8 |
| 30 | 4 | 4 | 4 | 4 | 3 | 4 |
| 60 | | 8 | | 7 | 7 | |
| 120 | 15 | 16 | 15 | 14 | 13 | 13 |
| 180 | | 24 | | 20 | 19 | |
| 240 | 31 | 31 | 31 | 26 | 24 | 25 |
| 360 | 46 | 45 | 45 | 37 | 36 | 37 |
| 480 | 60 | 58 | 59 | 48 | 46 | 47 |
| 600 | 72 | | 70 | | | 56 |
| 720 | 83 | | 80 | | | 65 |
| 840 | 93 | | 89 | | | 72 |
| 960 | 100 | | 95 | | | 79 |

TABLE 25
Dissolution profiles. Formula Y

| | Basket | Basket |
|---|---|---|
| Intervals | 100 rpm | 100 rpm |
| (min.) | Water | Gastric fluid |
| 30 | 7 | 6 |
| 60 | | 14 |
| 120 | 34 | 29 |
| 180 | | |
| 240 | 66 | 57 |
| 360 | 89 | 78 |
| 480 | | |
| 600 | 103 | |
| 720 | 104 | |

TABLE 26
Dissolution profiles. Formula W

| | Basket | Basket |
|---|---|---|
| Intervals | 100 rpm | 100 rpm |
| (min.) | Water | Gastric fluid |
| 30 | 4 | 3 |
| 60 | | 7 |
| 120 | 16 | 14 |
| 240 | 34 | 29 |
| 360 | 52 | 45 |
| 600 | 78 | |
| 720 | 88 | |
| 840 | 95 | |
| 960 | 101 | |

As the results show, release rates of drug are unaffected by pH as the viscosity of the gel which forms on the tablet surface and the rate of hydration are relatively independent of the pH environment. However, when ionic salts are used in the dissolution medium they can compete with the polymer and affect the dissolution rate of drug.

### Example 11

Plasma Concentration of N-(2-propylpentanoyl)glycinamide and of N-(2-propylpentanoyl)glycine After Administration.

Formulations A, B, and C were prepared as described in Example 1.

Two tablets of formulation A (2×500 mg active pharmaceutical ingredient) were simultaneously administered to each of 18 healthy male Caucasian volunteers. Plasma concentrations of N-(2-propylpentanoyl)glycinamide and of a major metabolite, N-(2-propylpentanoyl)glycine of each of the volunteers were regularly analyzed at 0.25, 0.5, 1.0, 1.5, 2, 4, 6, 8, 10, 12, 14, 16 and 24 hours.

The trial was then repeated with formulations B and C. The results of the trial were averaged and the mean plasma concentrations after administration of each of the formulations are depicted in FIGS. 1 and 2.

TABLE 27
Cₘₐₓ and Tₘₐₓ of N-(2-propylpentanoyl) glycinamide after
administering Formulations A, B, and C

| | Cₘₐₓ | | | Tₘₐₓ | | |
|---|---|---|---|---|---|---|
| | (μg/ml) | | | (μg/ml) | | |
| Subject | Formulation A | Formulation B | Formulation C | Formulation A | Formulation B | Formulation C |
| 1 | 10.98 | 7.22 | 22.83 | 4.00 | 4.00 | 2.00 |
| 2 | 9.85 | 8.54 | 32.06 | 6.00 | 16.00 | 0.25 |
| 3 | 9.77 | 7.39 | 22.67 | 10.00 | 16.00 | 2.00 |
| 4 | 9.39 | 7.91 | 19.48 | 12.00 | 16.00 | 0.50 |
| 5 | 10.12 | 6.07 | Not | 6.00 | 14.00 | Not |
| | | | Available | | | Available |
| 6 | 12.89 | 7.64 | 34.33 | 4.00 | 4.00 | 0.50 |
| 7 | 10.85 | 7.61 | 29.96 | 12.00 | 6.00 | 1.00 |
| 8 | 9.65 | 7.44 | 21.26 | 6.00 | 16.00 | 0.50 |
| 9 | 10.95 | 8.03 | 20.39 | 6.00 | 14.00 | 1.50 |
| 10 | 8.32 | 6.48 | 21.52 | 6.00 | 6.00 | 1.00 |
| 11 | 9.18 | 6.78 | 17.19 | 14.00 | 14.00 | 4.00 |
| 12 | 8.86 | 8.53 | 21.28 | 6.00 | 14.00 | 0.50 |
| 13 | 13.89 | 8.33 | 17.59 | 6.00 | 24.00 | 4.00 |
| 14 | 11.37 | 7.58 | 18.18 | 6.00 | 6.00 | 2.00 |
| 15 | 9.92 | 6.84 | 46.52 | 4.00 | 12.02 | 0.50 |
| 16 | 12.14 | 9.56 | 33.48 | 4.00 | 4.00 | 0.50 |
| 17 | 10.12 | 7.76 | 34.91 | 6.00 | 6.00 | 0.50 |
| 18 | 11.60 | 8.40 | 19.77 | 4.00 | 14.00 | 0.50 |
| N | 18 | 18 | 17 | 18 | 18 | 17 |
| Mean | 10.55 | 7.67 | 25.50 | 6.78 | 11.45 | 1.28 |
| SD | 1.44 | 0.84 | 8.22 | 3.08 | 5.73 | 1.19 |
| Min | 8.32 | 6.07 | 17.19 | 4.00 | 4.00 | 0.25 |
| Median | 10.12 | 7.63 | 21.52 | 6.00 | 14.00 | 0.50 |
| Max | 13.89 | 9.56 | 46.52 | 14.00 | 24.00 | 4.00 |

Cₘₐₓ is the maximum measured plasma concentration of N-(2-porpylpentanoyl) glycinamide after administration.
Tₘₐₓ is the time at which the maximum concentration of N-(2-porpylpentanoyl) glycinamide was measured.

As seen in FIG. 1, formulations A and B maintain a mean plasma concentration of N-(2-propylpentanoyl)glycinamide which is stable from 4 hours after administration to 16 hours after administration. In addition, in formulations A and B, mean Tₘₐₓ occurs after 6 hours, whereas in formulation C, mean Tₘₐₓ occurs before 2 hours.

As seen in table 27, the Cₘₐₓ after administration of formulations A and B did not exceed 14 μg/ml in any of the volunteers. However, the mean Cₘₐₓ after administration of formulation C was 25.5 μg/ml. Administration of formulations A or B may eliminate unwanted side-effects which are caused as a result of dosage peaks present in immediate release formulations such as formulation C.

TABLE 28
Cₘₐₓ and Tₘₐₓ of N-(2-propylpentanoyl) glycine after
administering Formulations A, B, and C

| | Cₘₐₓ | | | Tₘₐₓ | | |
|---|---|---|---|---|---|---|
| | (μg/ml) | | | (μg/ml) | | |
| Subject | Formulation A | Formulation B | Formulation C | Formulation A | Formulation B | Formulation C |
| 1 | 1.36 | 0.90 | 3.16 | 4.00 | 4.00 | 2.00 |
| 2 | 1.27 | 1.27 | 2.68 | 4.00 | 23.88 | 0.50 |
| 3 | 1.18 | 0.81 | 2.71 | 10.00 | 8.00 | 2.00 |
| 4 | 1.11 | 0.99 | 2.24 | 12.00 | 16.00 | 1.50 |
| 5 | 1.30 | 0.70 | | 4.00 | 12.00 | |
| 6 | 1.24 | 0.72 | 2.47 | 6.00 | 4.00 | 1.50 |
| 7 | 0.90 | 0.62 | 1.84 | 4.00 | 24.00 | 1.50 |
| 8 | 1.04 | 0.82 | 2.14 | 10.00 | 12.00 | 0.50 |
| 9 | 0.97 | 0.76 | 2.00 | 4.00 | 14.00 | 1.50 |
| 10 | 1.05 | 0.75 | 2.62 | 4.00 | 6.00 | 1.00 |
| 11 | 0.90 | 0.75 | 1.75 | 14.00 | 14.00 | 4.00 |
| 12 | 1.05 | 1.07 | 2.48 | 6.00 | 12.00 | 1.50 |
| 13 | 1.62 | 0.90 | 1.85 | 4.03 | 24.00 | 4.00 |
| 14 | 1.18 | 0.83 | 2.12 | 6.00 | 8.00 | 2.00 |
| 15 | 0.88 | 0.63 | 2.11 | 4.00 | 12.02 | 1.00 |
| 16 | 1.25 | 1.08 | 2.71 | 4.00 | 4.00 | 1.50 |
| 17 | 1.01 | 0.80 | 2.50 | 4.00 | 6.00 | 0.50 |
| 18 | 1.42 | 0.97 | 2.11 | 6.00 | 14.00 | 2.00 |
| N | 18 | 18 | 17 | 18 | 18 | 17 |
| Mean | 1.15 | 0.85 | 2.32 | 6.11 | 12.11 | 1.68 |
| SD | 0.20 | 0.17 | 0.38 | 3.18 | 6.66 | 1.01 |
| Min | 0.88 | 0.62 | 1.75 | 4.00 | 4.00 | 0.50 |
| Median | 1.15 | 0.82 | 2.24 | 4.02 | 12.00 | 1.50 |
| Max | 1.62 | 1.27 | 3.16 | 14.00 | 24.00 | 4.00 |

Cₘₐₓ is the maximum measured plasma concentration of N-(2-porpylpentanoyl) glycine after administration.
Tₘₐₓ is the time at which the maximum concentration of N-(2-porpylpentanoyl) glycine was measured.

N-(2-propylpentanoyl)glycine is one of the major metabolites of N-(2-propylpentanoyl)glycamine.

As seen in FIG. 2, formulations A and B maintain a mean plasma concentration of N-(2-propylpentanoyl)glycine which is stable from 4 hours after administration to 16 hours after administration. In addition, in formulations A and B, mean Tₘₐₓ occurs after 6 hours, whereas in formulation C, mean Tₘₐₓ occurs before 2 hours.

As seen in table 28, the Cₘₐₓ after administration of formulations A and B did not exceed 1.62 μg/ml in any of the volunteers. However, the mean Cₘₐₓ after administration of formulation C was 3.16 μg/ml. Administration of formulations A or B may eliminate unwanted side-effects which are caused as a result of dosage peaks present in immediate release formulations such as formulation C.

Discussion

In humans, neuropathic pain tends to be chronic. The same is true for epilepsy. In addition, epilepsy and neuropathic pain are diseases that require long term therapy. For most of the established drugs currently available for the treatment of these diseases, the required dosage must be administered several times daily. This results in compliance problems and fluctuations in plasma concentrations, which may lead to subtherapeutic and potentially toxic levels of the drug.

Development of sustained release formulations of anti neuropathic pain drugs and antiepileptic agents may improve the therapy of epileptic and/or neuropathic pain patients. The sustained release formulations of the present invention satisfy this pressing need.

In the present invention, the hydroxypropyl methyl cellulose is not part of the granule composition but is compressed with the granules into the final controlled release tablet. The formulations of the subject invention have the distinct advantage of allowing one to vary the desired dissolution profile of the resulting tablet without requiring one to remake the granule composition. Thus, according to the present invention, one can manufacture granules of the active material in bulk and then vary the dissolution profile of the resulting tablets by varying the amount and type of hydroxypropyl methyl cellulose added to the mixture. In addition, the present invention does not require that specific sizes of the granules be selected for the resulting tablets. Consequently, the process of manufacture presented above is significantly easier to implement than a process in which the hydroxypropylmethyl cellulose is part of the granule composition.

In addition, as described earlier, N-(2-Propylpentanoyl)glycinamide is difficult to work with due to its “lamination and compression” characteristics. Thus, the subject invention provides the unexpected result of using a filler and two types of hydroxypropylmethyl cellulose to improve the compression characteristics while simultaneously slowing down the drug release profile. As illustrated in Example 4, the tablets manufactured according to the subject invention are also extremely stable.

Furthermore, as illustrated in the examples, the use of two types of hydroxypropylmethyl cellulose yields tablets which release the drug at a steady rate over time, yet another advantage of the formulations of the subject invention.

Although the plasma concentration results in Example 11 are all based on administration of a single, 1000 mg dose of N-(2-propylpentanoyl)glycinamide, a linear pharmacokinetic response is expected in patients upon administration of other doses of similar formulation. Such a response is expected based on the work of Blotnick et al. with related compounds in phase I studies in which the pharmacokinetics were shown to be dose-independent (Blotnick et al., “The Disposition of Valproyl Glycinamide and Valproyl Glycine in Rats” (1997) Pharmaceutical Research 14(7): 873-878).

What is claimed is:

1. A sustained release solid dosage form comprising the following components:
a) a uniform admixture of:
(i) an active ingredient selected from the group consisting of valproic sodium acid, a pharmaceutically acceptable salt or ester of valproic acid, divalproex sodium, valpromide and a compound having the structure: 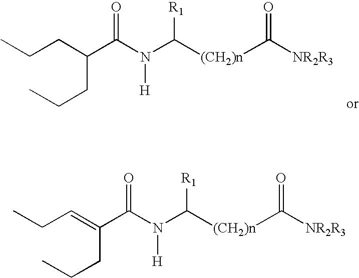
wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and
(ii) a binder; and
b) a hydroxypropylmethyl cellulose.

2. The solid dosage form of claim 1, wherein the solid dosage form is a tablet.

3. The solid dosage form of claim 1 or 2, wherein the uniform admixture of component a) further comprises a filler.

4. The solid dosage form of claim 3, wherein the filler comprises a microcrystalline cellulose.

5. The solid dosage form of claim 1 or 2, wherein the hydroxypropylmethyl cellulose comprises 19%-24% by weight methoxyl substituent, 7%-12% by weight hydroxyproproxyl substituent and has a particle size distribution such that at least 99% of the hydroxypropylmethyl cellulose passes through a No. 40 US standard sieve, has an apparent viscosity of 78-117 millipascal-seconds (nominal value 98 mPa.s) by rotation and 80-120 cP (nominal value 100 cP) by Ubbelhode, at a concentration of 1% by weight in water at 20° C., and has a pH in the range 5.5-8.0.

6. The solid dosage form of claim 5, wherein at least 90% of the hydroxypropylmethyl cellulose passes through a No. 100 US standard sieve.

7. The solid dosage form of claim 1 or 2, further comprising as additional components a filler, a lubricant and a flow agent.

8. The solid dosage form of claim 1 or 2, wherein the binder of component a)(ii) comprises hydroxypropyl cellulose.

9. The solid dosage form of claim 1 or 2, further comprising a different hydroxypropylmethyl cellulose as a component.

10. The solid dosage form of claim 3, further comprising as additional components a filler, a lubricant and a flow agent.

11. The solid dosage form of claim 10, further comprising a different hydroxypropylmethyl cellulose as a component.

12. The solid dosage form of claim 9 or 11, wherein the different hydroxypropylmethyl cellulose comprises 19-24% by weight methoxyl substituent, 7-9% by weight hydroxypropoxyl substituent, has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C., has a pH in the range 5.5-8.0 and has a particle size distribution such that at least 99% of the hydroxypropylmethyl cellulose passes through a No. 40 US standard sieve.

13. The solid dosage form of claim 12, wherein at least 90% of the hydroxypropylmethyl cellulose passes through a No. 100 US standard sieve.

14. The solid dosage form of claim 7, wherein
the filler comprises a microcrystalline cellulose, anhydrous dicalcium phosphate, lactose, methylcellulose, carboxymethylcellulose, calcium carbonate, calcium sulfate kaolin, sodium chloride, powdered cellulose, sucrose, mannitol or a combination of two or more of the foregoing;
the lubricant comprises magnesium stearate, sodium stearyl fumarate, hydrogenated castor oil, hydrogenated soybean oil, polyethylene glycol or a combination of two or more of the foregoing; and
the flow agent comprises a colloidal fumed silica, or colloidal silicon dioxide.

15. The solid dosage form of claim 14 wherein
the filler comprises a microcrystalline cellulose, anhydrous dicalcium phosphate, lactose or a combination of two or more of the foregoing;
the lubricant comprises magnesium stearate, sodium stearyl fumarate or a combination thereof; and
the flow agent comprises a colloidal fumed silica.

16. The solid dosage form of claim 1 or 2 wherein the active ingredient is a compound having the structure: 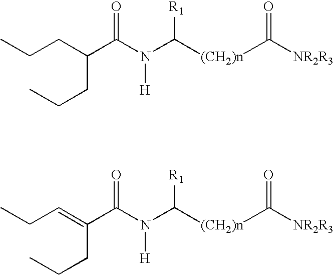
or
wherein R₁, R₂, and R₃ are independently the same or different and are hydrogen, a C₁-C₆ alkyl group, an aralkyl group, or an aryl group, and n is an integer which is greater than or equal to 0 and less than or equal to 3.

17. The solid dosage form of claim 16, wherein the active ingredient is N-(2-Propylpentanoyl)glycinamide.

18. A sustained release solid dosage form comprising the following components:
a) a uniform admixture of:
(i) N-(2-Propylpentanoyl)glycinamide; and
(ii) a binder;
b) a hydroxypropylmethyl cellulose; and
c) a different hydroxypropylmethyl cellulose.

19. The solid dosage form of claim 18, wherein the solid dosage form is a tablet.

20. The solid dosage form of claim 18 or 19, comprising a filler, a lubricant and a flow agent as additional components and wherein the uniform admixture of component a) further comprises a filler.

21. The solid dosage form of claim 20, wherein
the binder of component a)(ii) comprises hydroxypropyl cellulose;
the filler of component a) comprises a microcrystalline cellulose;
the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.;
the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;
the filler component comprises a microcrystalline cellulose, anhydrous dicalcium phosphate, lactose or a combination of two or more of the foregoing;
the lubricant component comprises magnesium stearate, sodium stearyl fumarate or a combination thereof; and
the flow agent component comprises a colloidal fumed silica.

22. The solid dosage form of claim 21, comprising the following components:
a) a uniform admixture of:
(i) from 50 mg/solid dosage form to 1000 mg/solid dosage form of N-(2-propylpentanoyl) glycinamide,
(ii) from 1 mg/solid dosage form to 100 mg/solid dosage form hydroxypropyl cellulose; and
(iii) from 1 mg/solid dosage form to 200 mg/solid dosage form microcrystalline cellulose;
b) from 10 mg/solid dosage form to 300 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;
c) from 10 mg/solid dosage form to 300 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;
d) from 1 mg/solid dosage form to 300 mg/solid dosage form microcrystalline cellulose, anhydrous dicalcium phosphate, lactose or a combination of two or more of the foregoing;
e) from 0.1 mg/solid dosage form to 20 mg/solid dosage form of magnesium stearate, sodium stearyl fumarate or a combination thereof; and
f) from 0.1 mg/solid dosage form to 15 mg/solid dosage form a colloidal fumed silica.

23. The solid dosage form of claim 21, comprising the following components:
a) a uniform admixture of:
(i) from 500 mg/solid dosage form to 850 mg/solid dosage form of N-(2-propylpentanoyl) glycinamide,
(ii) from 25 mg/solid dosage form to 75 mg/solid dosage form hydroxypropyl cellulose; and
(iii) from 50 mg/solid dosage form to 150 mg/solid dosage form microcrystalline cellulose;
b) from 100 mg/solid dosage form to 300 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;
c) from 20 mg/solid dosage form to 150 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;
d) from 20 mg/solid dosage form to 100 mg/solid dosage form microcrystalline cellulose, anhydrous dicalcium phosphate, lactose or a combination of two or more of the foregoing;
e) from 2 mg/solid dosage form to 20 mg/solid dosage form of magnesium stearate, sodium stearyl fumarate or a combination thereof; and
f) from 0.5 mg/solid dosage form to 5 mg/solid dosage form a colloidal fumed silica, per 1 gram solid dosage form.

24. The solid dosage form of any one of claims 22 or 23, wherein at least 90% of the hydroxypropylmethyl cellulose of component b), of component c), or of both component b) and c) passes through a No. 100 US standard sieve.

25. The solid dosage form of claim 23, wherein
the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

26. The solid dosage form of claim 23, comprising the following components:
a) a uniform admixture of
(i) 500 mg/solid dosage form N-(2-Propylpentanoyl)glycinamide,
(ii) 50 mg/solid dosage form hydroxypropyl cellulose; and
(iii) 100 mg/solid dosage form microcrystalline cellulose;
b) 150 mg/solid dosage form of hydroxypropylmethyl cellulose having 19%-24% by weight methoxyl substituent, 7%-12% by weight hydroxylproproxyl substituent and has a particle size distribution such that at least 99% of the hydroxypropylmethyl cellulose passes through a No. 40 US standard sieve;
c) 60 mg/solid dosage form of a different hydroxypropylmethyl cellulose having 19%-24% by weight methoxyl substituent, 7%-12% hydroxylproproxyl substituent and has a particle size distribution such that at least 99% of the hydroxypropylmethyl cellulose passes through a No. 40 US standard sieve;
d) 20 mg/solid dosage form lactose;
e) 4.5 mg/solid dosage form magnesium stearate; and
f) 1 mg/solid dosage form colloidal fumed silica.

27. The solid dosage form of claim 26, wherein at least 90% of the hydroxypropylmethyl cellulose of component b), of component c), or of both component b) and c) passes through a No. 100 US standard sieve.

28. The solid dosage form of claim 26, wherein
the hydroxypropylmethyl cellulose of component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
the hydroxypropylmethyl cellulose of component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

29. A hard compressed tablet comprising a uniform admixture of the following components:
a) N-(2-Propylpentanoyl)glycinamide;
b) a hydroxypropylmethyl cellulose; and
c) a different hydroxypropylmethyl cellulose.

30. The tablet of claim 29, wherein
the hydroxypropylmethyl cellulose component b) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve; and
the hydroxypropylmethyl cellulose component c) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

31. The tablet of any one of claims 29 or 30, wherein at least 90% of the hydroxypropylmethyl cellulose of component b), of component c), or of both component b) and c) passes through a No. 100 US standard sieve.

32. The tablet of claim 30, wherein
the hydroxypropylmethyl cellulose component b) has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
the hydroxypropylmethyl cellulose component c) has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

33. The tablet of claim 29, further comprising a filler, lubricant and flow agent as additional components.

34. The tablet of claim 33, wherein
the filler comprises a microcrystalline cellulose, anhydrous dicalcium phosphate, lactose or a combination of two or more of the foregoing;
the lubricant comprises sodium stearyl fumarate; and
the flow agent comprises a colloidal fumed silica.

35. The tablet of claim 34, comprising a uniform admixture of the following components:
a) from 100 mg/tablet to 1000 mg/tablet N-(2-Propylpentanoyl)glycinamide;
b) from 10 mg/tablet to 300 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;
c) from 10 mg/tablet to 300 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;
d) from 1 mg/tablet to 300 mg/tablet a microcrystalline cellulose, anhydrous dicalcium phosphate, lactose or a combination of two or more of the foregoing;
e) from 0.1 mg/tablet to 20 mg/tablet sodium stearyl fumarate; and
f) from 0.1 mg/tablet to 15 mg/tablet a colloidal fumed silica.

36. The tablet of claim 34, comprising a uniform admixture of the following components:
a) from 400 mg/tablet to 1000 mg/tablet N-(2-Propylpentanoyl)glycinamide;
b) from 100 mg/tablet to 300 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;
c) from 20 mg/tablet to 150 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;
d) from 10 mg/tablet to 60 mg/tablet a microcrystalline cellulose, anhydrous dicalcium phosphate, lactose or a combination of two or more of the foregoing;
e) from 2 mg/tablet to 20 mg/tablet sodium stearyl fumarate; and
f) from 5 mg/tablet to 15 mg/tablet a colloidal fumed silica,
per 1 gram tablet.

37. The tablet of claim 36, comprising a uniform admixture of the following components:
a) 500 mg/tablet N-(2-Propylpentanoyl)glycinamide;
b) 150 mg/tablet of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C.;
c) 60 mg/tablet of hydroxypropylmethyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.;
d) 20 mg/tablet lactose;
e) 10 mg/tablet sodium stearyl fumarate; and
f) 10 mg/tablet colloidal fumed silica.

38. A composition in granulate form comprising a uniform admixture of:
(i) an active ingredient selected from the group consisting of valproic sodium acid, a pharmaceutically acceptable salt or ester of valproic acid, divalproex sodium, valpromide and a compound having the structure: 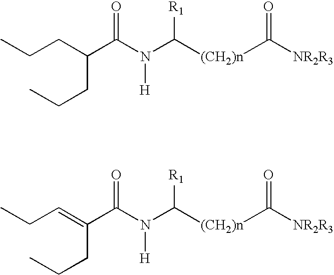
or
wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and
(ii) a hydroxypropyl cellulose.

39. The composition of claim 38, wherein the active ingredient comprises a compound having the structure: 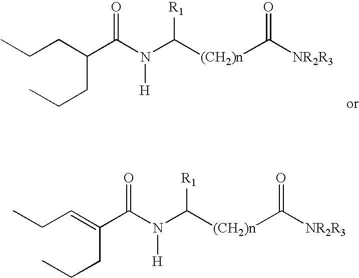
wherein R₁, R₂, and R₃ are independently the same or different and are hydrogen, a C₁-C₆ alkyl group, an aralkyl group, or an aryl group, and n is an integer which is greater than or equal to b and less than or equal to 3.

40. The composition of claim 38, wherein the active ingredient comprises valproic sodium acid, a pharmaceutically acceptable salt or ester of valproic acid, divalproex sodium or valpromide.

41. A tablet comprising the granulate of claim 38 as a component.

42. The tablet of claim 41, wherein the granulate further comprises a filler.

43. The tablet of claim 41, further comprising a hydroxypropylmethyl cellulose as a component.

44. The tablet of claim 41, further comprising as additional components a filler, a lubricant and a flow agent.

45. The tablet of claim 43, further comprising as additional components a filler, a lubricant and a flow agent.

46. The tablet of claim 43, further comprising a different hydroxypropylmethyl cellulose as a component.

47. The tablet of claim 43, wherein
the hydroxypropylmethyl cellulose has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

48. The tablet of claim 47, wherein at least 90% of the hydroxypropylmethyl cellulose passes through a No. 100 US standard sieve.

49. The tablet of claim 47, wherein
the hydroxypropylmethyl cellulose has an apparent viscosity of 78-117 millipascal-seconds (nominal value 98 mPa.s) by rotation and 80-120 cP (nominal value 100 cp) by Ubbelhode, at a concentration of 1% by weight in water at 20° C.

50. The tablet of claim 46, wherein
the different hydroxypropylmethyl cellulose has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

51. The tablet of claim 50, wherein at least 90% of the hydroxypropylmethyl cellulose passes through a No. 100 US standard sieve.

52. The tablet of claim 50, wherein
the different hydroxypropylmethyl cellulose has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

53. The tablet of claim 42, wherein the filler in the granulate is a microcrystalline cellulose.

54. The tablet of claim 45, wherein
the filler comprises a microcrystalline cellulose, anhydrous dicalcium phosphate, lactose or a combination of two or more of the foregoing;
the lubricant comprises magnesium stearate, sodium stearyl fumarate or a combination thereof; and
the flow agent comprises a colloidal fumed silica.

55. A sustained release tablet comprising a compound having the structure: 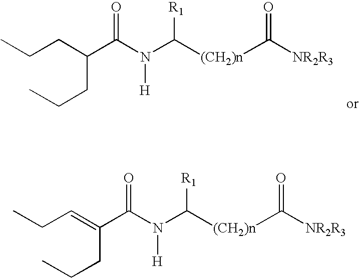
wherein R₁, R₂, and R₃ are independently the same or different and are hydrogen, a C₁-C₆ alkyl group, an aralkyl group, or an aryl group, and n is an integer which is greater than or equal to 0 and less than or equal to 3.

56. The sustained release tablet of claim 55, wherein the compound is N-(2-propylpentanoyl)glycinamide.

57. A method of treating neuropathic pain in a subject in need of such treatment comprising administering to the subject a therapeutically effective dose of the solid dosage form of any one of claims 1-28 or the tablet of any one of claims 29-37 or 41-56 in order to thereby treat the neuropathic pain in the subject.

58. A method of treating a headache disorder in a subject in need of such treatment comprising administering to the subject a therapeutically effective dose of the solid dosage form of any one of claims 1-28 or the tablet of any one of claims 29-37 or 41-56 in order to thereby treat the headache disorder in the subject.

59. A method of treating epilepsy in a subject in need of such treatment comprising administering to the subject a therapeutically effective dose of the solid dosage form of any one of claims 1-28 or the tablet of any one of claims 29-37 or 41-56 in order to thereby treat epilepsy in the subject.

60. A method of controlling seizures in a subject suffering from epilepsy comprising administering to the subject a therapeutically effective dose of the solid dosage form of any one of claims 1-28 or the tablet of any one of claims 29-37 or 41-56 in order to thereby control the seizures in the subject.

61. A method of treating pain in a subject in need of such treatment comprising administering to the subject a therapeutically effective dose of the solid dosage form of any one of claims 1-28 or the tablet of any one of claims 29-37 or 41-56 in order to thereby treat pain in the subject.

62. A method of pain prophylaxis in a subject in need of such treatment comprising administering to the subject a prophylactic dose of the solid dosage form of any one of claims 1-28 or the tablet of any one of claims 29-37 or 41-56 in order to thereby effect pain prophylaxis in the subject.

63. A method of treating mania in bipolar disorder in a subject in need of such treatment comprising administering to the subject a therapeutically effective dose of the solid dosage form of any one of claims 1-28 or the tablet of any one of claims 29-37 or 41-56 in order to thereby treat mania in bipolar disorder in the subject.

64. A method of attenuating bipolar mood swings in a subject suffering from bipolar disorder comprising administering to the subject a therapeutically effective dose of the solid dosage form of any one of claims 1-28 or the tablet of any one of claims 29-37 or 41-56 in order to thereby attenuate the bipolar mood swings in the subject.

65. A process for preparing the solid dosage form of claim 1 or 2, comprising the steps of:
a) admixing predetermined amounts of
(i) an active ingredient selected from the group consisting of valproic sodium acid, a pharmaceutically acceptable salt or ester of valproic acid, divalproex sodium, valpromide and a compound having the structure: 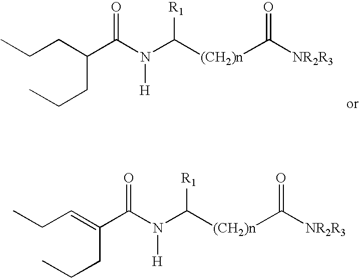
wherein R₁, R₂, and R₃ are independently the same or different and are hydrogen, a C₁-C₆ alkyl group, an aralkyl group, or an aryl group, and n is an integer which is greater than or equal to 0 and less than or equal to 3; and
(ii) a binder;
b) admixing the uniform mixture of step a) with a predetermined amount of a hydroxypropylmethyl cellulose; and
c) compressing the mixture of step b) to form the tablet.

66. The process of claim 65, wherein step b) further comprises admixing the uniform mixture with a predetermined amount of a different hydroxypropylmethyl cellulose.

67. The process of claim 66, wherein step b) further comprises admixing the uniform mixture with predetermined amounts of a filler, a lubricant and a flow agent.

68. The process of claim 67, wherein the flow agent comprises colloidal fumed silica.

69. The process of claim 67, wherein the filler comprises microcrystalline cellulose, anhydrous dicalcium phosphate, lactose or a combination of two or more of the foregoing.

70. The process of claim 69, wherein the filler comprises lactose.

71. The process of claim 67, wherein the lubricant comprises magnesium stearate or sodium stearyl fumarate or a combination thereof.

72. The process of claim 71, wherein the lubricant comprises magnesium stearate.

73. The process of claim 66, wherein
each hydroxypropylmethyl cellulose of step b) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

74. The process of claim 73, wherein at least 90% of the hydroxypropylmethyl cellulose passes through a No. 100 US standard sieve.

75. The process of claim 73, wherein
the first hydroxypropylmethyl cellulose has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
the second hydroxypropylmethyl cellulose has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

76. A process for preparing the hard compressed tablet of claim 29 comprising the steps of:
a) admixing predetermined amounts of N-(2-Propylpentanoyl)glycinamide, hydroxypropylmethyl cellulose, and a different hydroxypropylmethyl cellulose; and
b) compressing the mixture of step a) to form the hard compressed tablet.

77. The process of claim 76, wherein
each hydroxypropylmethyl cellulose of step a) has 19%-24% by weight methoxyl substituent, 7%-12% by weight hydroxylproproxyl substituent and has a particle size distribution such that at least 99% of the hydroxypropylmethyl cellulose passes through a No. 40 US standard sieve.

78. The process of claim 77, wherein at least 90% of the hydroxypropylmethyl cellulose passes through a No. 100 US standard sieve.

79. The process of claim 77, wherein
the hydroxypropylmethyl cellulose has an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
the different hydroxypropylmethyl cellulose has an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C.

80. The process of claim 76, wherein step a) further comprises admixing predetermined amounts of a filler, lubricant and flow agent as additional components.

81. The process of claim 80, wherein
the filler comprises microcrystalline cellulose, anhydrous dicalcium phosphate, lactose or a combination of two or more of the foregoing;
the lubricant comprises sodium stearyl fumarate; and
the flow agent comprises colloidal fumed silica.

82. A process for preparing the composition in granulate form of claim 38, comprising granulating a predetermined amount of valproic sodium acid, a pharmaceutically acceptable salt or ester of valproic acid, divalproex sodium, valpromide or a compound having the structure: 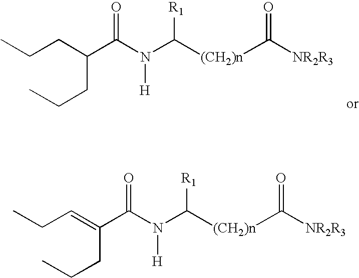
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, and a predetermined amount of hydroxypropyl cellulose to form the composition in granulate form.

83. A process for preparing a sustained release tablet comprising the steps of:
a) admixing the granules of claim 38 with predetermined amounts of a hydroxypropylmethyl cellulose; and
b) compressing the mixture of step a) to form the tablet.

84. The process of claim 83, wherein step a) further comprises admixing the granules with a predetermined amount of each of a different hydroxypropylmethyl cellulose, a filler, a lubricant and a flow agent.

85. The process of claim 84, wherein the flow agent comprises colloidal fumed silica.

86. The process of claim 84, wherein the filler comprises microcrystalline cellulose, anhydrous dicalcium phosphate, lactose or a combination of two or more of the foregoing.

87. The process of claim 86, wherein the filler is lactose.

88. The process of claim 84, wherein the lubricant comprises magnesium stearate or sodium stearyl fumarate or a combination thereof.

89. The process of claim 88, wherein the lubricant comprises magnesium stearate.

90. The process of claim 83, comprising the steps of:
a) admixing the granules with predetermined amounts of hydroxypropyl methyl cellulose having an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C., and hydroxypropyl methyl cellulose having an apparent viscosity of 78-117 millipascal-seconds (nominal value 98 mPa.s) by rotation and 80-120 cP (nominal value 100 cP) by Ubbelhode, at a concentration of 1% by weight in water at 20° C.; and
b) compressing the mixture of step a) to form the tablet.

91. The process of claim 90, wherein step a) further comprises admixing the granules with predetermined amounts of a flow agent, a filler, and a lubricant.

92. The process of claim 91 comprising the steps of
a) admixing the granules with
a predetermined amount of hydroxypropylmethyl cellulose with an apparent viscosity of 78-117 millipascal-seconds (nominal value 98 mPa.s) by rotation and 80-120 cP (nominal value 100 cP) by Ubbelhode, at a concentration of 1% by weight in water at 20° C. which results in tablets containing 150 mg/tablet;
a predetermined amount of hydroxypropyl methyl cellulose with an apparent viscosity of 6,138-9,030 millipascal-seconds (nominal value 7382 mPa.s) by rotation and 11,250-21,000 cP (nominal value 15,000 cP) by Ubbelhode at a concentration of 1% by weight in water at 20° C. which results in tablets containing 60 mg/tablet;
a predetermined amount of lactose which results in tablets containing 20 mg/tablet;
a predetermined amount of magnesium stearate which results in tablets containing 4.5 mg/tablet; and
a predetermined amount of a colloidal fumed silica which results in tablets containing 1 mg/tablet; and
b) compressing the mixture of step a) to form the tablet.

93. Use of an active ingredient selected from the group consisting of valproic sodium acid, a pharmaceutically acceptable salt or ester of valproic acid, divalproex sodium, valpromide and a compound having the structure: 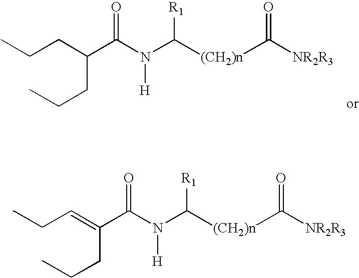
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in treating a headache disorder in a subject.

94. Use of an active ingredient selected from the group consisting of valproic sodium acid, a pharmaceutically acceptable salt or ester of valproic acid, divalproex sodium, valpromide and a compound having the structure: 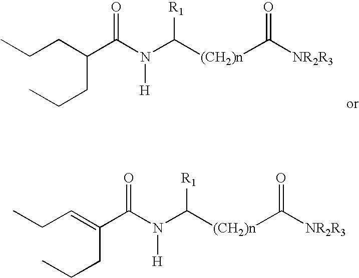
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing sustained release solid dosage form of any one of claims 1-28 or tablet of any one of claims 29-37 or 41-56 for use in treating neuropathic pain in a subject.

95. Use of an active ingredient selected from the group consisting of valproic sodium acid, a pharmaceutically acceptable salt or ester of valproic acid, divalproex sodium, valpromide and a compound having the structure: 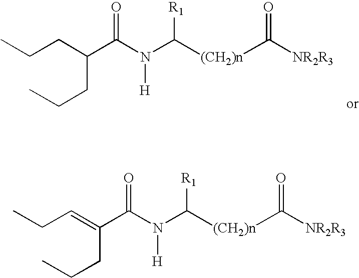
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in treating epilepsy in a subject.

96. Use of an active ingredient selected from the group consisting of valproic sodium acid, a pharmaceutically acceptable salt or ester of valproic acid, divalproex sodium, valpromide and a compound having the structure: 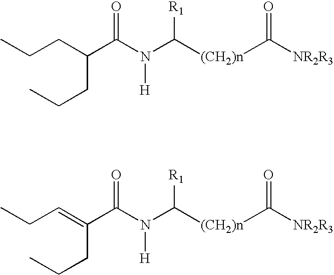
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in controlling seizures in a subject suffering from epilepsy.

97. Use of an active ingredient selected from the group consisting of valproic sodium acid, a pharmaceutically acceptable salt or ester of valproic acid, divalproex sodium, valpromide and a compound having the structure: 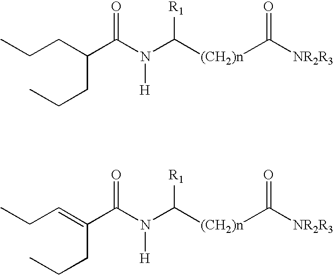
or
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in treating mania in bipolar disorder in a subject.

98. Use of an active ingredient selected from the group consisting of valproic sodium acid, a pharmaceutically acceptable salt or ester of valproic acid, divalproex sodium, valpromide and a compound having the structure: 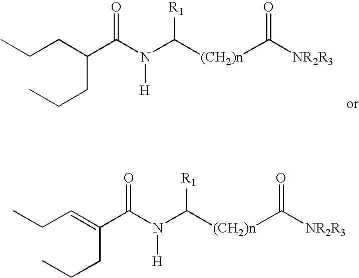
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in attenuating bipolar mood swings in a subject suffering from bipolar mood disorder.

99. Use of an active ingredient selected from the group consisting of valproic sodium acid, a pharmaceutically acceptable salt or ester of valproic acid, divalproex sodium, valpromide and a compound having the structure: 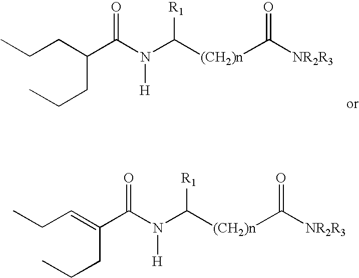
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in treating pain in a subject.

100. Use of an active ingredient selected from the group consisting of valproic sodium acid, a pharmaceutically acceptable salt or ester of valproic acid, divalproex sodium, valpromide and a compound having the structure: 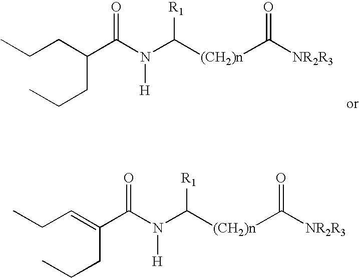
wherein R₁, R₂, and R₃ are independently the same or different and are hydrogen, a C₁-C₆ alkyl group, an aralkyl group, or an aryl group, and n is an integer which is greater than or equal to 0 and less than or equal to 3, for manufacturing a sustained release solid dosage form of any one of claims 1-28 or tablet of any one of claims 29-37 or 41-56 for use in effecting pain prophylaxis in a subject.

101. The sustained release solid dosage form of any one of claims 1-28 or tablet of any one of claims 29-37 or 41-56 for use in treating a headache disorder in a subject.

102. The sustained release solid dosage form of any one of claims 1-28 or tablet of any one of claims 29-37 or 41-56 for use in treating neuropathic pain in a subject.

103. The sustained release solid dosage form of any one of claims 1-28 or tablet of any one of claims 29-37 or 41-56 for use in treating epilepsy in a subject.

104. The sustained release solid dosage form of any one of claims 1-28 or tablet of any one of claims 29-37 or 41-56 for use in controlling seizures in a subject suffering from epilepsy.

105. The sustained release solid dosage form of any one of claims 1-28 or tablet of any one of claims 29-37 or 41-56 for use in treating mania in bipolar disorder in a subject.

105. The sustained release solid dosage form of any one of claims 1-28 or tablet of any one of claims 29-37 or 41-56 for use in attenuating bipolar mood swings in a subject suffering from bipolar disorder.

106. The sustained release solid dosage form of any one of claims 1-28 or tablet of any one of claims 29-37 or 41-56 for use in treating pain in a subject.

107. The sustained release solid dosage form of any one of claims 1-28 or tablet of any one of claims 29-37 or 41-56 for use in effecting pain prophylaxis in a subject.

108. A controlled release oral unit dose composition comprising N-(2-propylpentanoyl)glycinamide and at least one pharmaceutically acceptable carrier, wherein the composition when orally ingested by a human subject, induces a peak blood plasma level of N-(2-propylpentanoyl)glycinamide between 4 and 24 hours after ingestion of a single oral unit dose.

109. The controlled release oral unit dose composition of claim 108, wherein the composition when orally ingested by a human subject, induces a peak blood plasma level of N-(2-propylpentanoyl)glycinamide between 4 and 12 hours after ingestion of a single oral unit dose.

110. The controlled release oral unit dose composition of claim 109, wherein the composition when orally ingested by a human subject, induces a peak blood plasma level of N-(2-propylpentanoyl)glycinamide between 6 and 12 hours after ingestion of a single oral unit dose.

111. The controlled release oral unit dose composition of claim 110, wherein the composition when orally ingested by a human subject, induces a peak blood plasma level of N-(2-propylpentanoyl)glycinamide between 6 and 8 hours after ingestion of a single oral unit dose.

112. The controlled release oral dose composition of any one of claims 108 to 111, wherein the peak blood plasma level of N-(2-propylpentanoyl)glycinamide is from 0.5 micrograms/ml to 16 micrograms/ml per a 1000 mg dose of N-(2-propylpentanoyl)glycinamide in the composition.

113. The controlled release oral dose composition of claim 108, wherein the composition when orally ingested by a human subject, induces a peak blood plasma level of N-(2-propylpentanoyl)glycine in the human subject from 0.5 μg/mL to 1.7 μg/mL per a 1000 mg dose of N-(2-propylpentanoyl)glycinamide in the composition.

114. A controlled release oral dose composition comprising N-(2-propylpentanoyl)glycinamide and a pharmaceutically acceptable carrier, wherein the composition when orally ingested by a human subject, induces a peak blood plasma level of N-(2-propylpentanoyl)glycinamide of 0.5 μg/mL to 16 μg/mL per a 1000 mg dose in the composition.

115. A controlled release oral dose composition comprising N-(2-propylpentanoyl)glycinamide and a pharmaceutically acceptable carrier, wherein the composition when orally ingested by a human subject, induces a peak blood plasma level of N-(2-propylpentanoyl)glycine of 0.5 μg/mL to 1.7 μg/mL per a 1000 mg dose of N-(2-propylpentanoyl) glycinamide in the composition.

116. A method of inducing in a human subject a peak blood plasma level of N-(2-propylpentanoyl)glycinamide between 4 and 24 hours after administration of N-(2-propylpentanoyl)glycinamide, comprising administering to the human subject a controlled release oral unit dose composition comprising N-(2-propylpentanoyl)glycinamide and at least one pharmaceutically acceptable carrier, which composition induces a peak blood plasma level of N-(2-propylpentanoyl)glycinamide between 4 and 24 hours after administration of a single oral unit dose.

117. The method of claim 116, wherein the peak blood plasma level of N-(2-propylpentanoyl)glycinamide occurs between 4 and 12 hours after administration.

118. The method of claim 116, wherein the peak blood plasma level of N-(2-propylpentanoyl)glycinamide is 0.5 μg/mL to 16 μg/mL per 1000 mg dose of N-(2-propylpentanoyl)glycinamide in the composition.

119. The method of any one of claims 116-118, wherein the administration to the human subject of a controlled release oral unit dose composition comprising N-(2-propylpentanoyl)glycinamide and at least one pharmaceutically acceptable carrier induces a peak blood plasma level of N-(2-propylpentanoyl)glycine in the human subject from 0.5 μg/mL to 1.7 μg/mL upon administration of a single 1000 mg dose of N-(2-propylpentanoyl)glycinamide.

120. The method of any one of claims 116-119, wherein the controlled release oral dose composition is the solid dosage form of any one of claims 18-28 or the tablet of any one of claims 29-37 or 41-56.
